(19) United States
(12) Patent Application Publication
(10) Pub. No.: US 2025/0281238 A1
(43) Pub. Date: Sep. 11, 2025

(54) METHODS AND ARRANGEMENTS FOR CORRECTION PATH ADJUSTMENT FOR FIXATORS

(71) Applicants: Smith & Nephew, Inc., Memphis, TN (US); Smith & Nephew Orthopaedics AG, Zug (CH); Smith & Nephew Asia Pacific PTE. Limited, Singapore (SG)

(72) Inventors: Andrew Phillip Noblett, Bartlett, TN (US); Johnny R. Mason, Bartlett, TN (US)

(73) Assignees: Smith & Nephew, Inc., Memphis, TN (US); Smith & Nephew Orthopaedics AG, Zug (CH); Smith & Nephew Asia Pacific PTE. Limited, Singapore (SG)

(21) Appl. No.: 18/854,618

(22) PCT Filed: May 25, 2023
(86) PCT No.: PCT/US2023/023476

§ 371 Date: Oct. 7, 2024

Related U.S. Application Data
(60) Provisional application No. 63/345,963, filed on May 26, 2022.

(51) Int. Cl. A61B 34/10; A61B 34/00

(52) U.S. Cl. CPC A61B 34/10; A61B 34/25; A61B 2034/104; A61B 2034/105; A61B 2034/107

(57) ABSTRACT

Logic may interact with a user to generate or modify a code block of adjustments for a prescription or interact with the user via two-dimensional or three-dimensional image(s) to generate or modify a correction path of the treatment plan for a bone fixator. Logic may generate the prescription based on a deformity correction associated with a code block or based on a deformity correction identified by the user by modifying existing waypoints and/or adding new waypoints to the correction path. Logic may generate a display of an image of a fixed and a moving bone segment connected to the bone fixator and adjust the display to show a state of the bone deformity and the bone fixator at a point in time of the prescription selected by the user. And logic may display the remaining bone deformity for correction for the user during generation or modification of the correction path.

### CROSS-REFERENCE TO RELATED APPLICATIONS

This is a non-provisional of, and claims the benefit of the filing date of, U.S. provisional patent application No. 63/345,963, filed May 26, 2022, entitled “METHODS AND ARRANGEMENTS FOR CORRECTION PATH ADJUSTMENT FOR FIXATORS,” the entirety of which application is incorporated by reference herein.

### FIELD OF THE DISCLOSURE

The present disclosure relates generally to orthopedic devices, systems, and methods to adjust a correction path and particularly to adjust a correction path for bone fixator or bone segments connected to a bone fixator to generate a prescription of adjustments for a bone fixator to correct a bone deformity.

### BACKGROUND OF THE DISCLOSURE

Orthopedic surgeons must analyze a wide variety of deformities in which two or more bone segments are displaced or not aligned properly. Some simple deformities can be resolved acutely in clinic or in the operating room. Other ailments require careful planning and more prolonged treatment.

Also, many situations require the surgeons to perform an osteotomy to cut deformed bone segments apart so that a bone deformity can be corrected. After the osteotomy, the surgeons may apply an external fixator. After an external fixator is applied, the surgeon will need to analyze the patient's postoperative deformity to adjust the path of correction. It is standard practice for surgeons to take multiple medical images when analyzing orthopedic deformities. Typical practice involves capturing images of the involved bone segments in the frontal (AP) and sagittal (LAT) planes.

Orthopedic deformities are three dimensional problems and are typically described quantitatively with six deformity parameters, which can be measured with medical images and clinical evaluations. The deformity parameters are usually described as anteroposterior (AP) view translation, AP view angulation, sagittal (LAT) view translation, LAT view angulation, axial view translation, and axial view angulation. Deformity parameters may be evaluated from medical images, AP and Lateral radiographs or three-dimensional (3D) imaging modalities, and clinical evaluations.

Since deformity analysis can be complicated, many software solutions exist to assist surgeons with deformity analysis. Many external fixators also require software solutions to generate a schedule of hardware adjustments to correct the patient's bone deformity. Some software solutions include digital tools for preoperative deformity planning, postoperative deformity analysis, and hardware related parameters within the same system.

The rate and path of correction are critical to the healing process of external fixators. Most modern external fixator software solutions carefully control the rate and path of the correction in the generated schedule of hardware adjustments. If the rate and path of the correction are not carefully planned and controlled, complications such as impingement, over-stretching of anatomical structures, and/or preconsolidation of the regenerate bone can lead to patient pain, harm, interference with healing and even revision surgeries. An external fixator software solution offering precisely controlled customizability to the rate and path of correction would allow surgeons to further tailor hardware adjustment schedules to the needs of individual patients throughout treatment.

### SUMMARY OF THE DISCLOSURE

This Summary is provided to introduce a selection of concepts in a simplified form that are further described below in the Detailed Description. This Summary is not intended to identify key features or essential features of the claimed subject matter, nor is it intended as an aid in determining the scope of the claimed subject matter.

Some examples include methods and arrangements for a graphical user interface to adjust a correction path for a bone fixator. The methods and arrangements may include generating a display of an image of a fixed bone segment and a moving bone segment connected to the bone fixator. The display may represent a point in time of a prescription and may depict a state of the bone fixator at the point in time as well as a state of the deformity via a translation and an angulation of the moving bone segment relative to the fixed bone segment. The methods and arrangements may also include a first user interface element to interact with a user to select the point in time of the prescription, wherein a perspective view of the display is user definable. The methods and arrangements may also include adjusting the translation and the angulation of the moving bone segment relative to the fixed bone segment by the user graphically via user actions. The user actions may include at least one of dragging a point on the moving bone to a new waypoint to adjust the translation, the angulation, or a combination of the translation and the angulation of the moving bone segment or entering keystrokes to adjust the translation, the angulation, or a combination of the translation and angulation of the moving bone segment to the new waypoint.

In any preceding or subsequent example, the methods and arrangements may further include a second user interface element to interact with a user to select the perspective view of the display. In some examples, the image of a fixed bone segment and a moving bone segment includes two dimensional radiological images of the bone segments connected with the bone fixator. In some examples, the image of a fixed bone segment and a moving bone segment includes a three-dimensional image of the bone segments connected with the bone fixator.

In any preceding or subsequent example, the methods and arrangements may further include a third user interface element to describe the remaining deformity in response to identification of the new waypoint. In some examples the corrections necessary to fully correct the remaining deformity after a waypoint is introduced are applied automatically as a new waypoint. In some examples, the methods and arrangements may further include a fourth user interface element to describe hardware adjustments responsive to identification of the new waypoint. In some examples, the methods and arrangements may further include a fifth user interface element to describe a number of days associated with the prescription, wherein the number of days is updated responsive to identification of the new waypoint.

In any preceding or subsequent example, the new waypoint may update a final corrected state, wherein a partial deformity remains in the final corrected state after the update to the final corrected state. In other words, the final prescription may include the partial deformity. In some examples, the final prescription includes additional days of adjustment to correct the partial deformity. In any preceding or subsequent example, a granularity for selection of the point in time

includes selection of an adjustment in the prescription and, in some examples, the granularity for selection of the point in time includes selection of a day in the prescription.

In any preceding or subsequent example, the methods and arrangements may further include calculating a three-dimensional projection of two curves on two-dimensional images to generate one three-dimensional linear curve for adjustments of the correction path. In some examples, the methods and arrangements may further include generating adjustments based on user input of a nonlinear three-dimensional curve to generate three-dimensional linear adjustments for the correction path.

In any preceding or subsequent example, the correction path is limited to hardware constraints. In some examples, the image includes a three-dimensional model of the bone segments, a statistical model, or a three-dimensional medical image. In such examples, the three-dimensional medical model is based on a magnetic resonance image (MRI), a computerized tomography (CT) scan, AP and LAT radiographs, other x-ray images, other medical images, or a combination thereof.

Some examples include methods and arrangements for a code block interface to adjust a correction path for a bone fixator. The methods and arrangements may include interacting with a user to generate or modify a code block of adjustments for a prescription, a set of the code blocks including the code block to describe the prescription. Each code block of the set of code blocks may include a stage of adjustments for the prescription and each stage of adjustments may include simultaneous adjustments for the bone fixator. The methods and arrangements may also include generating the prescription for each code block based on a deformity correction associated with the code block, a maximum distraction rate associated with the code block, and a maximum rotation/angulation rate associated with the code block. In such examples, the prescription may include a number of days to perform a correction associated with the code block and hardware modification associated with the prescription. In such examples, generating the prescription accounts for hardware limitations/constraints and displaying each code block. The display of each code block may include the deformity correction associated with the code block, the maximum distraction rate associated with the code block, and the maximum rotation/angulation rate associated with the code block. The prescription may include the number of days to perform the correction associated with the code block and hardware modification associated with the correction of the code block. In such examples, the prescription may account for hardware limitations/constraints. Such examples may also display a first user interface element to describe a remaining deformity after correction via the set of code blocks.

In any preceding or subsequent example, the methods and arrangements may further include generating a display of an image of a fixed bone segment and a moving bone segment connected to the bone fixator, the display representing a point in time of a prescription, the display depicting a state of the bone fixator at the point in time and a state of the deformity via a translation and an angulation of the moving bone segment relative to the fixed bone segment, wherein a perspective view of the display is user definable.

In any preceding or subsequent example, generating the display includes generating the display with two images, a first image of a state of the deformity prior to correction by a selected stage and second image of a state of the deformity after correction by the selected stage. In some examples, the image includes a three-dimensional model of the bone segments, a statistical model, or a three-dimensional medical image, wherein the three-dimensional medical model is based on a magnetic resonance image (MRI), a computerized tomography (CT) scan, AP and LAT radiographs, other x-ray images, other medical images, or a combination thereof.

In any preceding or subsequent example, the methods and arrangements may further include a second user interface element to interact with a user to the perspective view of the display.

In any preceding or subsequent example, the image of a fixed bone segment and a moving bone segment includes two dimensional radiological images of the bone segments connected with the bone fixator.

In any preceding or subsequent example, the image of a fixed bone segment and a moving bone segment includes a three-dimensional image of the bone segments connected with the bone fixator.

In any preceding or subsequent example, the methods and arrangements may further include interacting with the user to update a final corrected state, wherein a partial deformity remains after the update to the final corrected state. In some examples, a final prescription includes the partial deformity at the final corrected state. In some examples, the final prescription includes additional days of adjustment to correct the partial deformity to reach the final corrected state.

In any preceding or subsequent example, the methods and arrangements may further include calculating one three-dimensional linear curve for adjustments of the correction path. In some examples, the correction path is limited by hardware constraints.

Examples of the present disclosure provide numerous advantages. For example, correction logic circuitry may advantageously include operations such as providing a graphical user interface to adjust a correction path for a bone fixator, graphically displaying the path of the bone segments during each adjustment of a prescription, graphically customizing the correction path by manipulating the displayed models, and clicking and dragging a point of the correction path in three-dimensional space to change the correction path of the resulting adjustment schedule.

Customizing correction paths may be simple or complex but does not have to be linear. Correction logic circuitry may advantageously include operations such as fine tuning the magnitude of movements without having to drag points around the display, breaking adjustments into correction steps/stages, automatically introduce new correction steps/stages to manage the remaining deformity in response to adjusting the correction path in a direction that adds to the deformity or does not fully solve the deformity, automatically defining the ending position of a correction step, automatically adding more correction steps/stages, pushing angular correction from one step to the another step, dividing angular correction between multiple correction steps, and/or the like.

Correction logic circuitry may advantageously include operations such as using code blocks with or separately from a graphical user interface to adjust a correction path on an image; organizing the deformity parameters into code blocks once each deformity parameter is defined with a magnitude and direction; correcting parameters according to the rate limits defined by the user; customizing the correction path by rearranging the code blocks into stages; correcting the remaining deformity in one or more additional steps/stages; plotting the correction path on three-dimensional or two dimensional images of the bone segments; generating code blocks based on manipulations of the correction path on the three-dimensional or two dimensional images of the bone segments; altering a rate of correction in millimeters per day (mm/day) and/or degrees per day (deg/day) for each correction step/stage; combining code blocks movement from one view or multiple views together; recording with the code blocks, the deformity being corrected as shown or as the movement of the bone segment/frame (opposite of the deformity); and/or the like.

Further features and advantages of at least some of the examples of the present disclosure, as well as the structure and operation of various examples of the present disclosure, are described in detail below with reference to the accompanying drawings.

### BRIEF DESCRIPTION OF THE DRAWINGS

By way of example, specific examples of the disclosed device will now be described, with reference to the accompanying drawings, in which:

FIG. 1A illustrates an example of is a system for treating a patient;

FIGS. 1B-F illustrate examples of anteroposterior (AP) view and lateral (LAT) view outline images of a tibia aligned and misaligned;

FIGS. 2A-H illustrate examples of a user interface to provide postoperative input data to correction logic circuitry;

FIG. 2I illustrates an example of a prescription with daily adjustments;

FIG. 3A-I illustrates an example of user interaction with a graphical interface having a three-dimensional (3D) image of a deformity to adjust a correction path of a prescription such as the prescription shown in FIG. 1I;

FIGS. 4A-E illustrate examples of user interaction with a graphical interface having code blocks to adjust a correction path of a prescription such as the prescription shown in FIG. 1I;

FIGS. 5A-B illustrate examples of flowcharts for user interaction with a graphical interface having one or more images and/or code blocks to adjust a correction path;

FIG. 5C illustrates an example of a flowchart to adjust a correction path of a prescription such as the prescription shown in FIG. 1I;

FIG. 6 depicts an example of a system including a multiple-processor platform, a chipset, buses, and accessories the server, HCP device, and the patient device shown in FIG. 1A; and

FIGS. 7-8 depict examples of a storage medium and a computing platform such as the server, HCP device, and the patient device shown in FIG. 1A and FIG. 6.

The drawings are not necessarily to scale. The drawings are merely representations, not intended to portray specific parameters of the disclosure. The drawings are intended to depict various examples of the disclosure, and therefore are not to be considered as limiting in scope. In the drawings, like numbering represents like elements.

### DETAILED DESCRIPTION

External fixators utilizing software to determine any hardware adjustments necessary to achieve the correction and, by extension, the correction path and correction rate of the fixator during treatment have been used clinically for decades. A surgeon enters parameters in the software to define the patient's deformity, the hardware applied to the patient, the hardware location(s) relative to the deformity, and the desired duration/distraction rate of the correction. Based on the surgeon's inputs the fixator software determines the final, corrected state of the patient's fixator and provides an adjustment schedule, which will allow the fixator to be controllably manipulated from the initial postoperative state to the final corrected state. The bone is divided into a fixed bone segment and a moving bone segment. During correction, the moving bone segment moves according to hardware adjustments, e.g., adjustments to the lengths of struts of the fixator. The path that the moving bone segment follows during the correction is referred to as the correction path. The schedule of hardware adjustments is referred to as the prescription.

Many fixator software programs dictate correction paths by prioritizing an even distribution of hardware adjustments during a correction. Such fixator programs calculate distances between the initial and final positions of a point on the moving bone segment and the initial and final states of the hardware. Some fixator programs calculate the distance a point on a specified anatomic structure of interest travels during the correction. No matter which type of point is used, the calculated travel distance of the point is divided by the maximum allowable correction rate in units of millimeters per day (mm/day) to determine the number of days for the correction. Angular correction rate parameters control the degrees of rotation per day of correction (deg/day) and may also be considered in the calculation of the number of days required for the correction. Such fixator programs distribute the hardware adjustment required to correct the deformity according to the calculated number of days required for the correction. In some programs the calculated movements of the bone segments from this algorithm may be checked against rate limiting inputs for all adjustments (maximum allowed translation per day and/or maximum allowable degrees of rotation per day). If any of the calculated adjustments exceed defined rate limits, then additional days may be added to the adjustment schedule to ensure that limits are always maintained. Such fixator programs determine correction paths determined by hardware adjustments but do not ensure the moving bone segment follows a linear path.

Other fixator programs dictate the correction path of a fixator according to the path of the anatomy. Such fixator programs calculate a line between the initial and final locations of a point on the moving bone segment (or a point on a specified anatomic structure as described above). Such fixator programs then calculate hardware adjustments so that the moving bone segment will follow the linear path within a tolerance band according to the specified rate of correction. If any of the calculated adjustments exceed defined rate limits, then additional days may be added to the adjustment schedule to ensure that limits are always maintained.

Some fixator programs allow users to adjust the correction path but in very limited ways. One example, common to fixator software programs is the ability for the surgeon to divide an adjustment schedule into two phases. The first phase corrects the specified axial translation deformity (lengthening or shortening) and the second phase corrects the remaining deformity. This allows the surgeon to distract the bone segments for clearance before correcting translation and angulation in other directions.

Some fixator programs offer more customization of the correction path through waypoints of correction. The waypoints build upon the axial translation first mode by allowing surgeons break a correction into multiple phases and correct specified amounts of angulation and translation within each phase. For such fixator programs, the surgeons must specify the numerical value and direction of the angulation or translation that is to be corrected in each phase. However, modern fixator programs that allow users to alter the correction path of an external fixator through waypoints and other means are not user friendly and are often overcomplicated.

Examples include systems and arrangements for correction path adjustment for bone fixators to determine a prescription of adjustments. In some examples, during the correction phase of treatment with a bone fixator, one bone segment is manipulated in relation to another bone segment. The manipulation may be guided by correction logic circuitry which may generate a prescription of adjustments to achieve a proper correction based on hardware, deformity, and rate defining inputs.

The correction logic circuitry may display one or more images of the bone segments and graphically display the correction path of the bone segments. The correction logic circuitry may allow the user to enter waypoints to the correction path, graphically or via keystrokes, on two or more two-dimensional images such as AP and LAT radiographs, other x-ray images, other medical images, or a combination thereof. The correction logic circuitry may calculate a three-dimensional projection of two curves on two-dimensional images to generate one three-dimensional curve representing the correction path of the fixator.

In some examples, the correction logic circuitry may display an image of the bone segments as a three-dimensional model of the bone segments or dowels, a statistical model, or a three-dimensional medical image. In some examples, the image may be a three-dimensional medical model based on a magnetic resonance image (MRI), a computerized tomography (CT) scan, AP and LAT radiographs, other x-ray images, other medical images, or a combination thereof.

In some examples, one bone segment is identified as a fixed bone segment and the other bone segment is identified as a moving bone segment. The correction logic circuitry may interact with a user graphically or via keystrokes to graphically manipulate a correction path on 2D images or a 3D image for correction and/or interact with a user via code blocks to define waypoints or stages of the correction path for deformity correction. Until the conception of examples discussed herein, no system has manipulated the correction path of a bone fixator or the connected bone segments, by graphically defining a curve and/or graphically defining waypoints or stages via code blocks.

Many examples may optionally curve fit waypoints to generate linear adjustments for the correction path from non-linear waypoints. In some examples, the correction logic circuitry may, based on user input of a nonlinear three-dimensional curve, generate three-dimensional linear adjustments for the correction path. In some examples, the correction logic circuitry may generate a display or display one or more images of bone segments to illustrate or display the deformity and the correction path to a user. In many examples, the one or more images may include a representation of the bone fixator including the transosseous elements that interconnect the bone fixator with the fixed bone segment and the moving bone segment.

The waypoints may define the correction path in stages of correction from an initial postoperative state to a final corrected state. The final corrected state may optionally leave some remaining deformity of the bone segments. In some examples any remaining deformity is applied to a new waypoint automatically, but the full correction may be overridden.

The code blocks are graphical elements that display the correction path in a series of one or more stages of deformity correction. Each of the code blocks may include deformity corrections in terms of deformity parameters such as AP view translation, AP view angulation, LAT view translation, LAT view angulation, Axial view translation, and Axial view angulation. The correction logic circuitry may generate hardware adjustments to achieve the correction defined by each code block.

The correction logic circuitry may also allow the user to define distinct parameters and/or user preferences for each code block (or stage of correction). For instance, each of the code blocks may be associated with an independent rate of translation and rate of rotation/angulation of the moving bone segment per day. In many examples, the user may interact with the correction logic circuitry to establish maximum rates of translation and rotation/angulation that apply to all the stages of correction also.

Along with the graphical display of the code blocks and/or correction path curves, the correction logic circuitry may display a remaining deformity to inform the user of the deformity parameters input or calculated from the initial deformity analysis of the bone segments that has not been accounted for by existing code blocks or customized correction path stages. For instance, the user may interact with the correction logic circuitry to generate an initial correction and thus prescription to solve a patient's deformity. The user may then use code blocks or correction path curves to modify the initially calculated correction by breaking the correction into stages, altering the magnitude and/or direction of the deformity being corrected in each stage, order the correction stages, or rate of correction stages. The correction logic circuitry may represent the resulting correction in one or more code blocks or correction stages depending on the example, and a user interface element of the display may show a remaining deformity that results from differences between the initial correction and the adjusted correction.

While many examples herein discuss and illustrate an exterior bone fixator for tibia and fibula fractures, examples are applicable to deformations or fractures of any orthopedic correction area. Furthermore, examples described herein focus primarily on a single fracture that separates a bone into two bone segments, but examples are not limited to a single fracture or osteotomy of, e.g., a tibia or fibula. Examples may address each pair of bone segments separately and the bone segments may be part of any bone. For instance, a tibia may be fractured into three bone segments, i.e., a first bone segment, a second bone segment, and a third bone segment. Such examples may identify the deformity of the first bone segment and the second bone segment and identify the deformity of the third bone segment with respect to the second bone segment.

Logic circuitry herein refers to a combination of hardware and code to perform functionality. For instance, the logic circuitry may include circuits such as processing circuits to execute instructions in the code, hardcoded logic, application specific integrated circuits (ASICs), processors, state machines, microcontrollers, and/or the like. The logic circuitry may also include memory circuits to store code and/or data, such as buffers, registers, random access memory modules, flash memory, and/or the like.

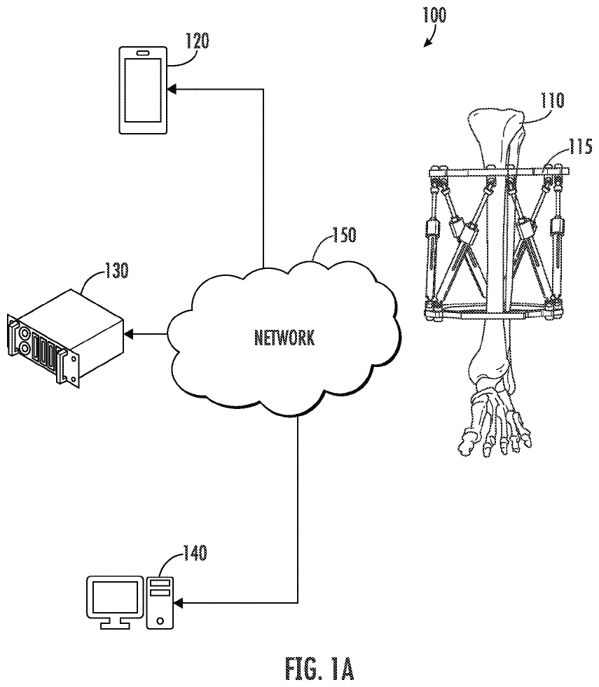

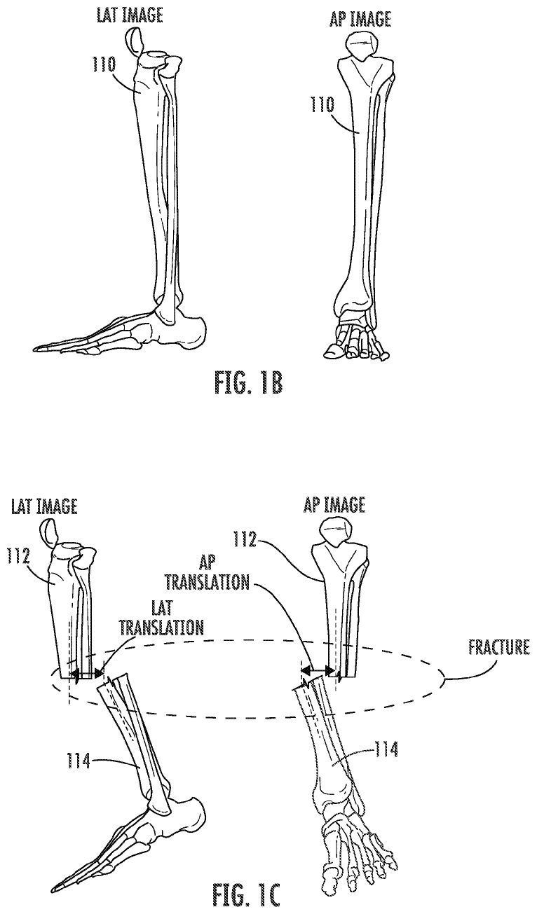

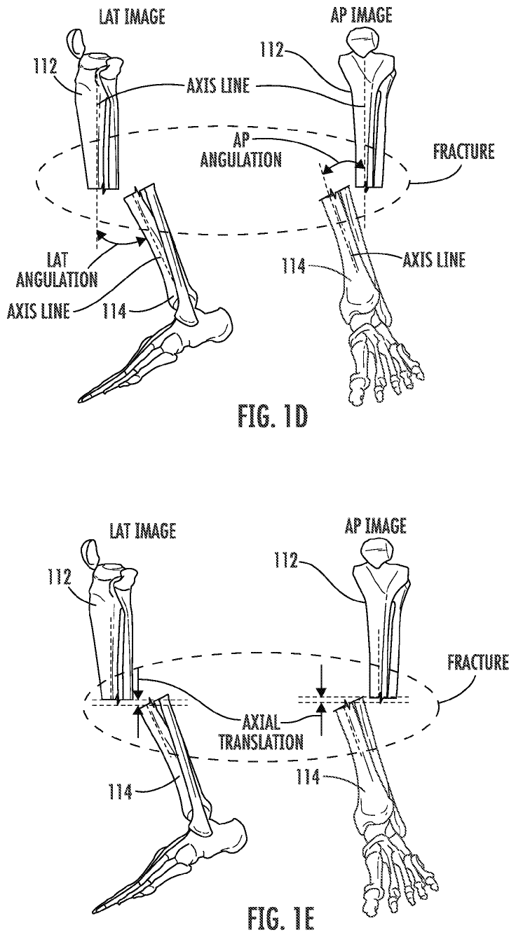

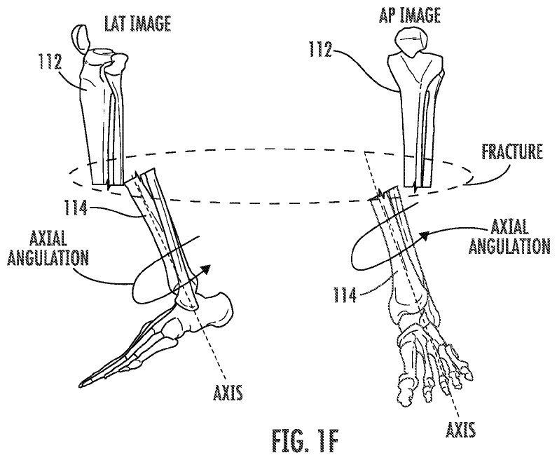

An example of a system 100 for treating a patient is illustrated in FIG. 1A. The system illustrated is only one example of a system that includes correction logic circuitry to generate and/or modify a correction path or treatment plan for a correction of a bone deformity of the bone 110 with a bone fixator 115. Other systems may use other types of orthopedic devices and/or processing circuitry to generate and/or modify a correction path for a correction of a bone deformity.

The system 100 may include the external fixator 115 configured to be coupled to a patient, a patient device 120 connected to a network 150, a server 130 connected to the network 150, and a Health Care Practitioner (HCP) device 140 connected to the network 150. The illustrated external fixator 115 may include, e.g., a six-axis external fixator. In other examples, an external fixator 115 may be any device capable of coupling to two or more bone segments of a bone 110 and moving or aligning the bone segments relative to one another.

The patient device 120 illustrated is a handheld wireless device. In other examples, a patient device may be any brand or type of electronic device capable of executing a computer program and outputting results to a patient. For example, and without limitation, the patient device 120 may be a smartphone, a tablet, a mobile computer, or any other type of electronic device capable of providing one or both of input and output of information. In some examples, the patient device 120 may couple with the network 150 via wired and/or wireless connections to facilitate use of the patient device 120 to display, implement, and/or provide feedback related to implementation of a prescription for the external fixator 115. In many examples, the server 130 and/or the HCP device 140 may transmit a prescription to the patient device 120 and/or updates for the prescription to the patient device 120 responsive to the feedback related to implementation of a prescription for the external fixator 115.

The network 150 may be one or more interconnected networks, whether dedicated or distributed. Non-limiting examples include personal area networks (PANs), local area networks (LANs), wide area networks (WANs), metropolitan area networks (MANs), private and/or public intranets, the Internet, cellular data communications networks, switched telephonic networks or systems, and/or the like. Connections to the network 150 may be continuous or may be intermittent, only providing for a connection when requested by a sending or receiving client device.

The server 130 is shown connected to the network 150 in FIG. 1A. The server 130 may be a single computing device in some examples or may itself be a collection of two or more computing devices and/or two or more data storage devices that collectively function to process data as described herein. The server 130, or any one or more of its two or more computing devices, if applicable, may connect to the network 150 through one or both of firewall and web server software and may include one or more databases. If two or more computing devices or programs are used, the devices may interconnect through a back-end server application or may connect through separate connections to the network 150. The server 130 or any component server device of the system may include integrated or separate computer readable media containing instructions to be executed by the server. For example, and without limitation, computer readable media may be any volatile or non-volatile media integrated into the server 130 such as a hard disc drive, random access memory (RAM), or non-volatile flash memory. Such computer readable media, once loaded into the server 130 as defined herein, may be integrated, non-transitory data storage media. In some examples, a server 130 may include a storage location for information that will be eventually used by the patient device 120, the server 130, and/or the HCP device 140.

When stored on the server 130, memory devices of the server 130, as defined herein, provide non-transitory data storage and are computer readable media containing instructions. Similarly, computer readable media may be separable from the server 130, such as a flash drive, external hard disc drive, tape drive, Compact Disc (CD), or Digital Versatile Disc (DVD) that is readable directly by the server 130 or in combination with a component connectable to the server 130.

In some examples, correction logic circuitry of the server 130 may communicate

with the HCP device 140 via, e.g., a web browser or other client software installed on the HCP device 140 (correction logic circuitry). The correction logic circuitry may facilitate interaction with a user such as an orthopedic surgeon to create or correct a correction path for a fixator such as the external fixator 115 to correct a deformity of the bone 110 based on a set of one or more images such as radiographs, preoperative user input data (optionally), postoperative input data, user preferences, and data in data structures such as one or more databases or libraries. In some examples, the correction logic circuitry of the server 130 may interact with the user graphically via the image(s) and/or via code blocks to create and/or adjust a correction path, divide the correction path into stages of correction, and/or the like. In other examples, the correction logic circuitry may reside on and may include, e.g., code for execution by a processor of the HCP device 140 so that a network 150 may not be required.

The one or more images may be a single image such as a radiograph of the bone 110 for a two-dimensional (2D) description of a deformity of the bone 110 and may include two 2D images or one 3D image for a three-dimensional description of the deformity. Additional medical imaging (e.g., magnetic resonance imaging (MRI), computed tomography (CT), x-ray, ultra-sound, etc.) can be used to create a three-dimensional (3D) model of the patient's bone 110 to analyze deformity parameters of the bone 110 and to facilitate generation of a postoperative prescription for the external fixator 115 for correcting the deformity of the bone 110. In some examples, the one or more images may include additional images if the code is part of a more complex software application that offers functionality in addition to the generation or modification of a postoperative prescription. For instance, a hexapod software application may use deformity parameters from a deformity analysis and additional inputs to determine a strut adjustment schedule or prescription for the external fixator 115. In some examples, the correction logic circuitry may use one or more or any combination of edge and image recognition software, x-ray markers, manual inputs, automated inputs, augmented reality systems, and sensor technologies to gather input data related to the bone deformity of the bone 110 as well as input data related to the hardware of the fixator installed such as the external fixator 115.

In some examples, the correction logic circuitry may include code executing on the HCP device 140 and on the server 130 and may include one or more databases operating on the server 130. The databases may include one or more data structures or libraries including multiple orthopedic devices for one or more different bones, fixations for the orthopedic devices, strut dimensions and adjustability, other hardware limitations/constraints, and/or the like.

The correction logic circuitry may interact with a user to upload one or more postoperative images of the bone 110 and obtain postoperative input data for determining the positions of an orthopedic device and transosseous elements with respect to the bone 110. The input data may include an anatomy of and deformity location for the bone 110 such as a left mid-shaft tibia and may, in some examples, provide a list from which the user may identify an orthopedic device attached to the bone 110 such as the external fixator 115 based on data accessible via a data structure such as a library. In some examples, the anatomy of the bone 110 may be included in the file name or metadata of the one or more images uploaded by the user.

The user may also provide user input data to describe the positions, angles, edge geometry, and relationship to the bone fragments (also referred to as bone segments or bone fragments) of transosseous elements, struts, rings, mounting hardware, and/or the like. This orientation and geometrical data may be input by the user, derived from analysis of medical images or models, or any combination of the two. Based on this input data, the correction logic circuitry may calculate the positions of the rings, transosseous elements, mounting hardware, struts, and, in some examples, neurovascular clusters, to avoid impingement on any of the structures by other structures along the correction path during the course of a prescription.

In many examples, the correction logic circuitry may display a 2D or 3D model of the orthopedic device, such as the external fixator 115, with the transosseous elements attached to the bone 110 and may provide for user interaction graphically or via keystrokes to create or adjust the correction path for the bone deformity. In some examples, the correction logic circuitry may also or alternatively interact with the user to create or adjust a display of code blocks representative of a prescription.

In many examples, the correction logic circuitry may present the image(s) with indications of the correction path and/or code blocks with indications of corrections of the deformity parameters in one or more correction steps/stages. For instance, the correction logic circuitry may illustrate the correction path for a particular day or point in time of the prescription with one or more waypoints. The one or more waypoints may include a first waypoint to demark a position of a point on one of the bone segments such as the moving bone segment of the bone 110 and a second waypoint illustrating the position to which an adjustment of the correction path may angulate, rotate, and/or translate the moving bone segment. In some examples, the correction logic circuitry may allow the user to drag a waypoint on a graphical display from one location to another location to identify where the user prefers the waypoint to be located. The correction logic circuitry may determine a change to a remaining deformity, change a user element that shows the remaining deformity accordingly, and determine if the new waypoint location causes an impingement via impingement analysis. In some examples, if the new waypoint location causes an impingement, an indication of the impingement is highlighted, e.g., with a color, in a note field, and/or the like. In some examples, the correction logic circuitry may not allow the new waypoint position to be added to the prescription if the waypoint position causes an impingement. In some examples, once a new waypoint is added or a current waypoint is moved to a new position, and the correction logic circuitry determines that the new waypoint position does not cause an impingement or violate any other rules or preferences established by the user, the user may add the new waypoint position to the correction path of the prescription. In some examples the correction logic circuitry may determine a best fit correction path curve to avoid impingement.

In some examples, the correction logic circuitry may include a user interface element to describe the current adjustment to a prescription based on user interaction with one or more of the waypoints. For instance, the user element may display the deformity correction prior to the user interaction, the deformity correction after the user interaction, the deformity that remains uncorrected after the current adjustment prior to the user interaction, the amount of the deformity remaining (uncorrected) after the user interaction, and/or the like.

In some examples, code blocks are displayed in addition to the image(s) on the user interface or are displayed in lieu of the image(s) of the bone 110 with the external fixator 115. A code block may represent a stage of correction that includes one or more adjustments. The code block may describe the deformity corrections in terms of the deformity parameters and/or in terms of angulations and translations of the changes made to the position of the moving bone segment. In some examples, the code block may also include indications of limits of angulation and/or translation per day of the prescription. In some examples, each code block may have independent limits and, in other examples, each stage of correction may assume the same maximum angulation and translation limits set by the user. Some examples may create a prescription with more than one adjustment per day. In some of these examples, the user may set a limit on the magnitude of the translation and/or angulation per adjustment. In some of these examples, the user may set a limit on the number of adjustments per day.

In some examples, the correction logic circuitry may show, in real-time, the remaining deformity correction based on the addition or modification of code blocks and/or the addition or modification of waypoints on the image(s). For examples that illustrate both the image(s) and the code blocks, a change made to waypoints in the images may also be reflected in real time in the corresponding code blocks. Furthermore, a change made to code blocks may also be reflected in real time in the corresponding waypoints in the images.

In many examples, the correction logic circuitry includes user interface elements that can be manipulated graphically or with keystrokes, to help the user determine new positions for waypoints and/or code block adjustments. The user interface elements may interact with a user, e.g., to adjust the perspective view of the bone 110 in a 3D image, to adjust the point in time (or day of adjustment) of a prescription illustrated by the image(s), to display a projection of a 3D curve of the correction path on a 3D image or on two or more 2D images, to display differences in deformity correction between the current adjustment and a new adjustment, and/or the like. In some examples, the correction logic circuitry may illustrate a current correction path and a revised correction path, based on interaction with a user, as points and/or curves on the image(s) of the bone 110.

In some examples, the correction logic circuitry may include user interface elements to interact with a user to show the progression of movement of the moving bone segment throughout a correction path of a prescription to the final corrected state of the bone 110. In some examples, the user may play the correction path of a prescription forward to show the progression along the correction path of the moving bone segment from the initial postoperative state to the final corrected state. In further examples, the user may also play the progression of the correction path for the prescription in reverse from the final corrected state to the initial postoperative state.

In some examples, the correction logic circuitry may perform an impingement analysis based on one or more correction paths for the struts, distal ring(s), transosseous elements coupled with the distal ring(s) and fixations. In some examples, the correction logic circuitry may determine movements required to achieve the final corrected state based on postoperative user inputs of the initial bone deformity and the final corrected state of the bone 110. In other examples, the correction logic circuitry may use a current prescription and/or a user modified prescription to perform impingement analyses for the external fixator 115.

Note that examples can use images captured from any angle or orientation and movements of bone segments may be defined in relation to the coordinate system implemented by the correction logic circuitry. Thus, references to vertical or horizontal movements relative to a 2D or 3D image may not reflect the actual components of such movements determined and stored by the correction logic circuitry unless properly oriented by the user. For instance, a vertical movement with respect to a particular image may represent movement along an x-axis, a y-axis, a z-axis, or any combination thereof, with respect to the coordinate system implemented by the correction logic circuitry. Thus, the correction logic circuitry may record such movements as a tuple or vector such as (x,y,z), where x, y, and z represent numbers indicative of movement in units such as millimeters or centimeters along the x-axis, y-axis, and z-axis, respectively. A movement of zero, in some examples, may represent no movement, a negative movement may represent movement in a first direction with respect to an axis, and a positive movement may represent movement in a second direction with respect to the axis.

AP and LAT views are common practice for radiographs of fractures and bone deformities, but examples are not limited to AP and LAT view images. Furthermore, as long as each of the images has a known scale, the images do have to be the same scale. The correction logic circuitry may translate or convert scales to a selected or default scale implemented by the correction logic circuitry and translate or convert movements associated with bone segments and struts in images to a coordinate system implemented by the correction logic circuitry.

Note that examples are not limited to the correction logic circuitry residing in the server 130. The correction logic circuitry may reside in whole or in part in the HCP device 140. The correction logic circuitry may reside in whole or in part in the server 130. Furthermore, the correction logic circuitry may reside partially in multiple compute servers and data storage servers managed by a management device and operating as the server 130. The correction logic circuitry may also or alternatively reside partially in multiple computers and/or storage devices such as the HCP device 140. Where the correction logic circuitry may reside partially in multiple computers, the correction logic circuitry may include management logic circuitry to manage multiple local and/or remote resources.

The HCP device 140 is shown connected to the network 150. The HCP device 140 illustrated is a desktop personal computer. In other examples, the HCP device 140 may be any brand or type of electronic device capable of executing a computer program and receiving inputs from or outputting information to a user. For example, and without limitation, the HCP device 140 may be a smartphone, a tablet computer, or any other type of electronic device capable of providing one or both of input and output of information. Such a device may provide a user interface for data input, waypoint, or code block modification, as well as communication with a patient, another HCP, or a device or system manufacturer.

An HCP device such as the HCP device 140 may be connected to the network 150 by any effective mechanism. For example, and without limitation, the connection may be by wired and/or wireless connection through any number of routers and switches. Data may be transmitted by any effective data transmission protocol. The HCP device 140 may include integrated or separate computer readable media containing instructions to be executed by the HCP device 140. For example, and without limitation, computer readable media may be any media integrated into the HCP device 140 such as a hard disc drive, RAM, or non-volatile flash memory. Such computer readable media once loaded into the HCP device 140 as defined herein may be integrated and non-transitory data storage media. Similarly, computer readable media may be generally separable from the HCP device 140, such as a flash drive, external hard disc drive, CD, or DVD that is readable directly by the HCP device 140 or in combination with a component connectable to the HCP device 140.

FIGS. 1B-1F illustrate LAT and AP images of an unfractured tibia, bone 110, and the same tibia fractured into a first bone segment 112 and a second bone segment 114. Each of FIGS. 1C-1F illustrate at least one of the deformity parameters on the LAT image and the AP image. Note that while the illustrations focus on the tibia and LAT and AP images, examples may process any other bone and any other viewing angle in a similar manner.

FIG. 1B illustrates an example of an AP and a LAT image of an unfractured tibia, bone 110. Note that the AP image provides a fontal view of the tibia and the LAT view provides a side view of the tibia.

FIG. 1C illustrates an example of the tibia bone 110 fractured into two bone segments, a first bone segment 112 and a second bone segment 114. As discussed therein, the first bone segment typically refers to the fixed bone segment if the processing involves a fixed bone segment. For instance, some examples fix the first bone segment, and all deformity parameters are determined based upon movement of the second (moving) bone segment to align the second bone segment with the first bone segment.

In FIG. 1C, the example may determine the LAT translation based on a horizontal translation of the second bone segment 114 to align the second bone segment with the first bone segment 112 on the LAT image. Similarly, the example may determine the AP translation based on a horizontal translation of the second bone segment 114 to align the second bone segment with the first bone segment 112 on the AP image.

FIG. 1D illustrates an example of the tibia bone 110 divided into two bone segments, a first bone segment 112 and a second bone segment 114 for purpose of illustrating the deformity parameters of LAT angulation and AP angulation. A way to illustrate and/or determine the LAT or AP angulation is to overlay a first axis line through the axis of the first bone segment 112, overlay a second axis line through the axis of the second bone segment 114, and measure the angle between the first and second axis lines. The angle between the first and second axis lines may be the LAT or AP angulation, depending on the view.

FIG. 1E illustrates an example of the tibia bone 110 fractured into two bone segments, a first bone segment 112 and a second bone segment 114 for purpose of illustrating the deformity parameter of axial translation. Many examples determine the axial translation as the vertical movement of either or both the first bone segment 112 and the second bone segment 114 to bring the two bone segments together. Many examples determine the final axial translation based on interaction with the user. For 2D deformity parameters, the final axial translation may be determined from a single image. For 3D deformity parameters, the final axial translation parameter may be determined after calculation of an axial translation for two or more images such as a LAT view and an AP view of the bone segments. Some examples may have a user define an origin one point on one bone segment and a corresponding point on the other bone segment such that translation may be defined as the component distances between the origin and corresponding points.

FIG. 1F illustrates an example of the tibia bone 110 fractured into two bone segments, a first bone segment 112 and a second bone segment 114 for purpose of illustrating the deformity parameter of axial angulation. The axial angulation is the rotation of the second bone segment 114 about the axis of the second bone segment 114 to align the second bone segment with the first bone segment 112. In many examples, the axial angulation is determined clinically.

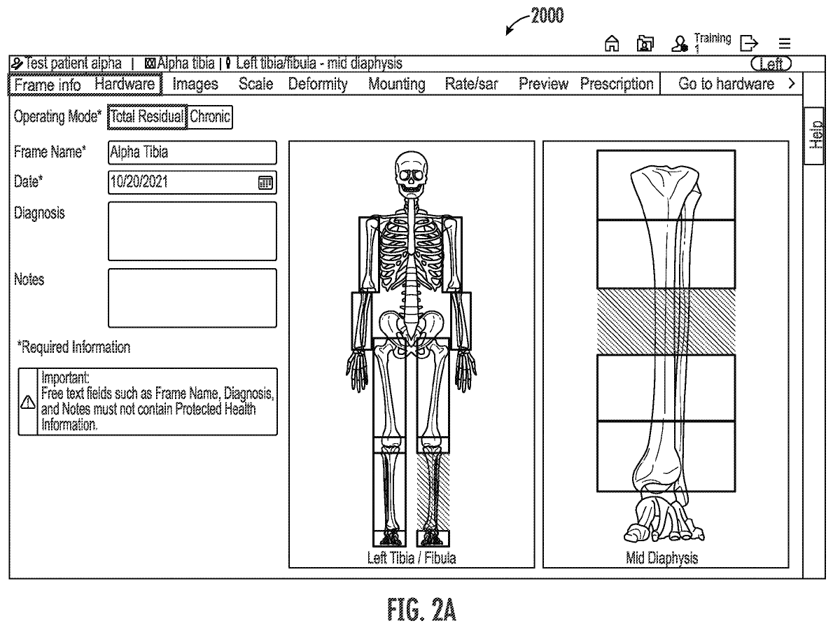

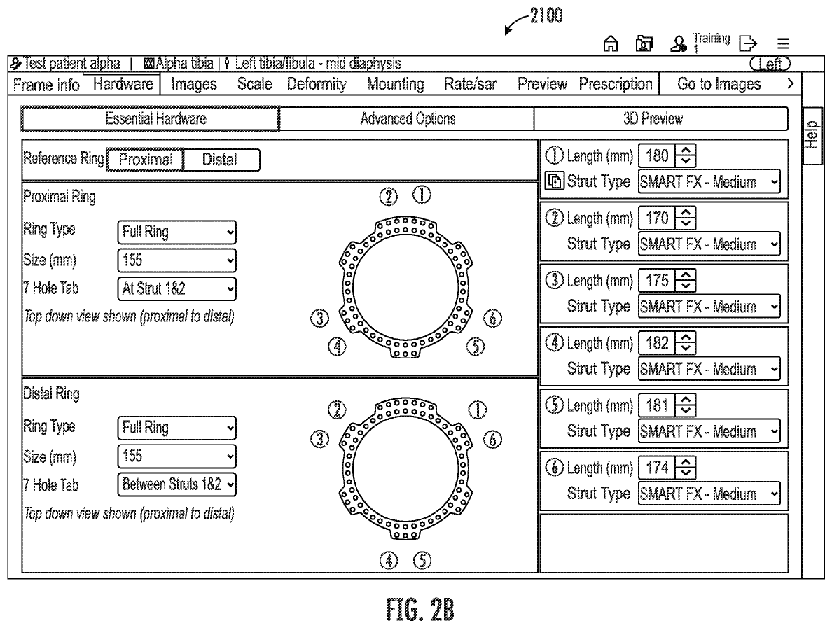

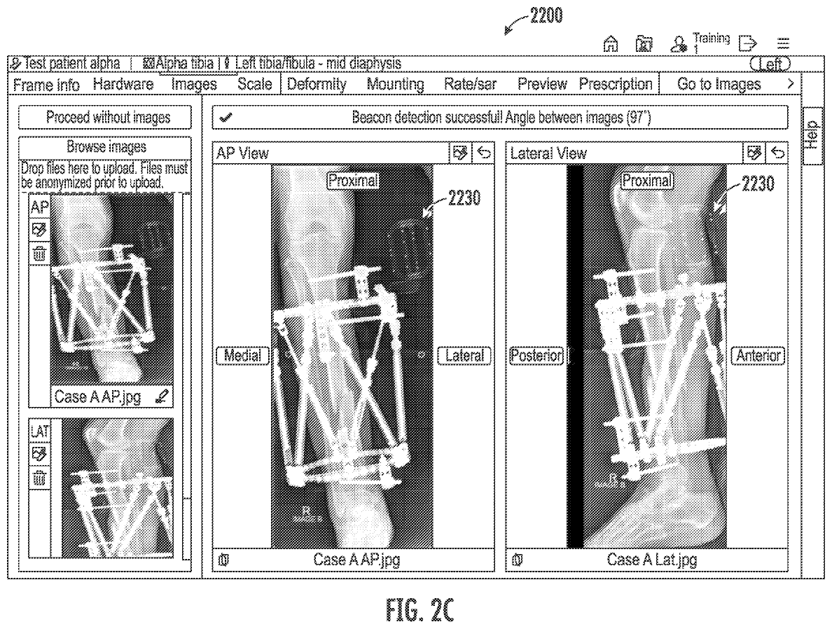

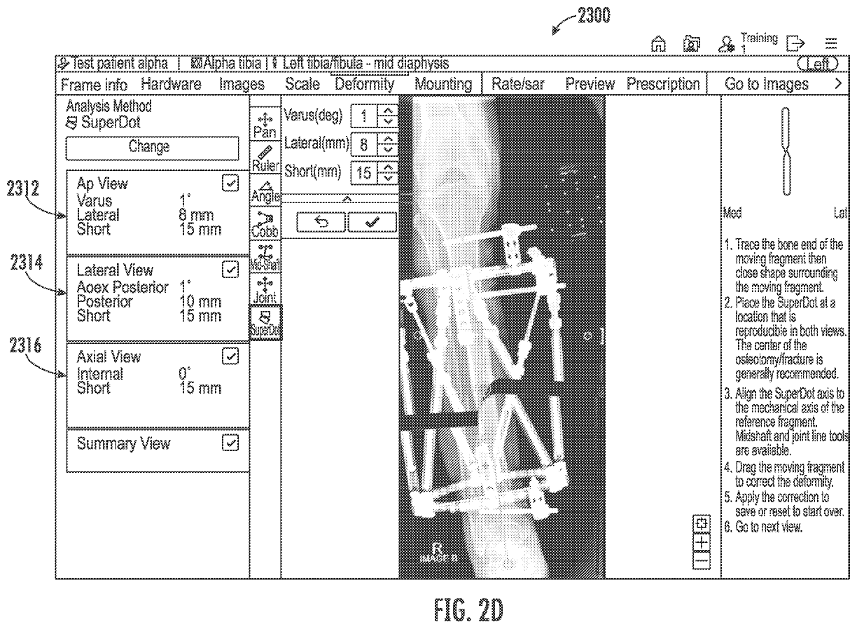

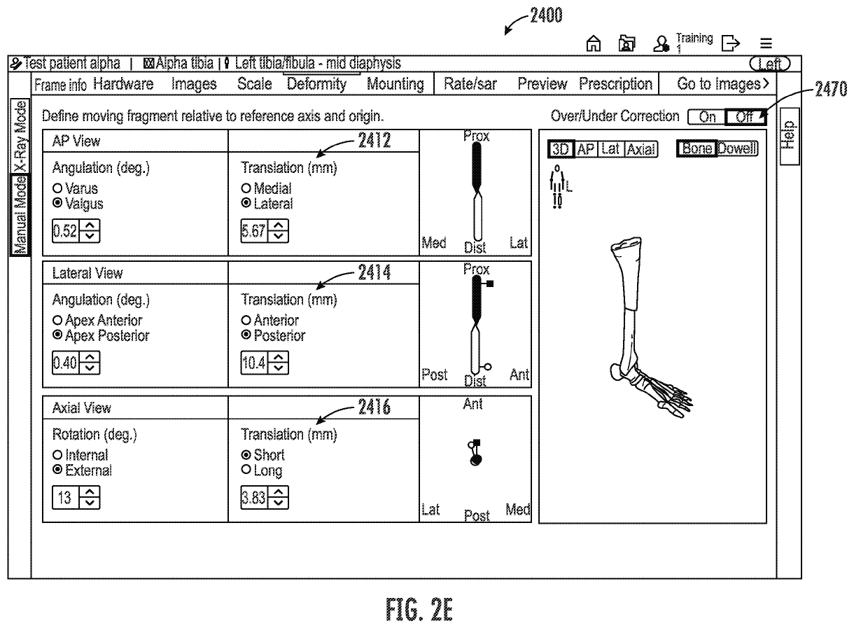

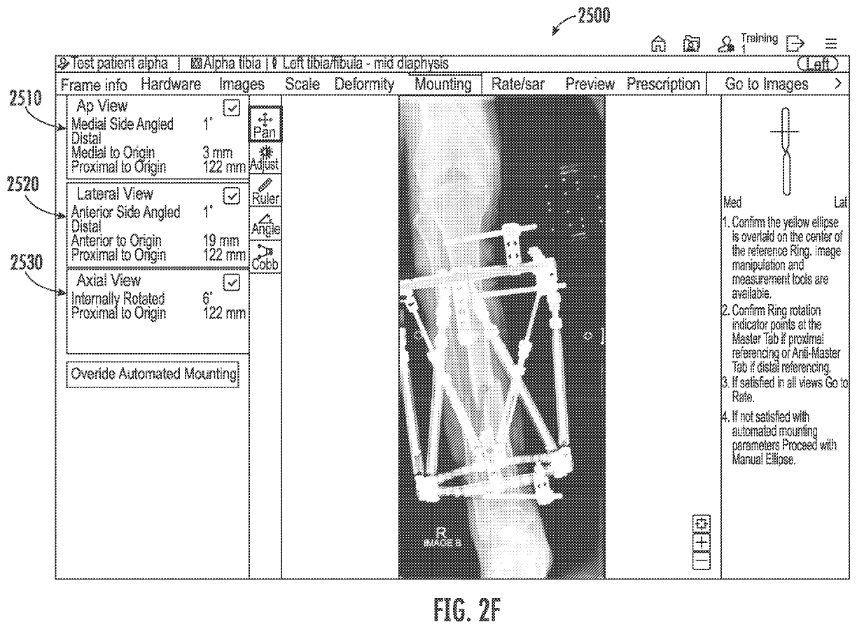

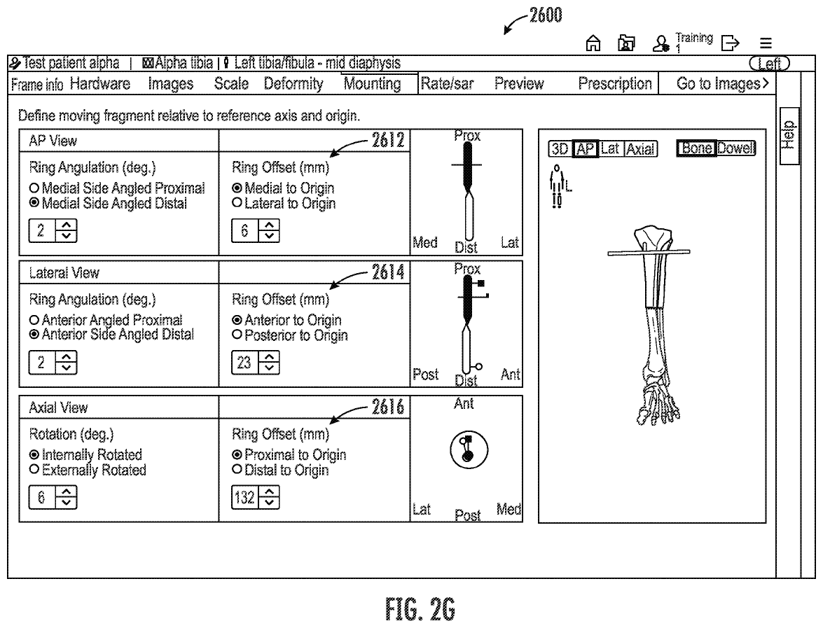

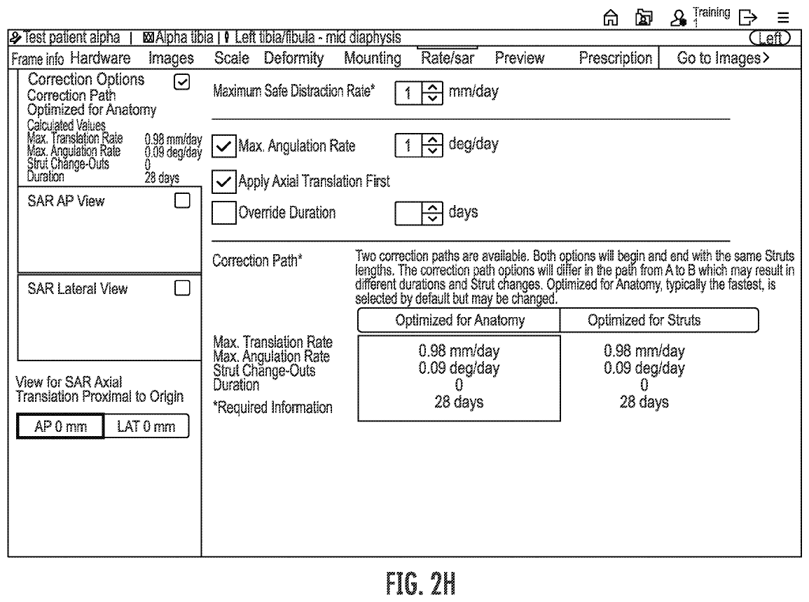

FIGS. 2A-H illustrate examples of a user interface to input data to correction logic circuitry such as the correction logic circuitry discussed in conjunction with FIGS. 1A-F.

FIG. 2A illustrates an example of a user interface 2000 of correction logic circuitry for user input data including information such as a file name, diagnosis, notes, general anatomical region of interest, and anatomical hand.

FIG. 2B illustrates an example of a user interface 2100 of correction logic circuitry for a user to input data about the bone fixator. In some examples, the user input data includes identification of the reference hardware component, such as a reference ring. In some examples the selection of a reference hardware component may determine the fixed and moving bone segments. The user input data may also include identification relevant hardware parameters such as for, e.g., the ring type, ring size, strut lengths, strut sizes, strut types, strut mount locations. In other examples, the number and/or the types of hardware may differ depending on the type of bone fixator.

In other examples, the user interface 2100 may also include additional data entry for the hardware such as the type, size, location, angle, and mounting hardware for the transosseous elements that attach the bone fixator to the bone segments. Such examples may include impingement analysis that includes transosseous elements, neurovascular structures, mounting hardware for the transosseous elements, and/or the like in addition to the struts and rings. Such examples may also include a database such as an electronic library of hardware components and dimensions such as the dimensions of the struts, rings, transosseous elements, mounting hardware for the transosseous elements, and/or the like.

In some examples, the edge geometry of the bone fixator is defined to allow for impingement analysis. In some examples, edge geometry is defined when the user selects the hardware components from a list via a data structure such as a library. In other examples, edge geometry must be input manually or defined on medical images (e.g., AP and Lateral radiographs) for relevant hardware components instead of or in addition to selection from a list. In some examples the edge geometry of hardware components may be defined in medical images automatically by the correction logic circuitry via edge detection algorithms, radiographic markers, and the like.

FIG. 2C illustrates an example of a user interface 2200 for correction logic circuitry to upload images for the bone deformity such as 2D AP and LAT radiological images. Note that, in some examples, the correction logic circuitry may allow the user to choose to upload and scale radiological images if radiological planning is desired or may allow the user to proceed without uploading images to define deformity parameters manually. The radiological planning may allow the user to identify the deformity and mounting parameters via graphical interaction with the radiological images.

FIG. 2D-E illustrate examples of a user interface 2300 or 2400 for the correction logic circuitry to obtain input data about the bone deformity.

FIG. 2D illustrates the user interface 2300 may allow the user to graphically identify the bone deformity in the AP view 2312, the Lateral view 2314, and the Axial view 2316 via the medical images (e.g., AP view and LAT view radiographs), and/or to manually enter the deformity.

FIG. 2E illustrates an example of a user interface 2400 of the correction logic circuitry that offers manual entry of the deformity parameters as an alternative to entry graphically via the radiological images. In the example the deformity parameters are manually defined in the AP view 2412, Lateral View 2414, and Axial View 2416.

The method of analyzing the deformity is unimportant if the deformity parameters can be related back to points on the bone segments and/or hardware. In some examples, like the examples shown inf FIG. 2D-E, the deformity parameters will be input as 2D components of the 3D deformity parameters (e.g., AP angulation, AP translation, LAT angulation, LAT translation, Axial angulation (rotation), and Axial translation). Other examples may directly capture or allow input of the 3D deformity parameters.

In some examples, the user interface 2400 may also include a user interface element 2470 to allow the user to select the option of over correction or under correction. If the over/under correction is set to disabled, the correction logic circuitry may generate a prescription to completely correct the deformity. In other words, the magnitude of all deformity parameters will be zero by the end of the prescription (after the final adjustment). In some examples, disabling over/under correction may result in the correction logic circuitry automatically generating additional steps, waypoints, or code blocks to a correction path modified by the user so that no deformity will remain at the end of the prescription. If the over/under correction is enabled, the correction logic circuitry may guide the user to correct the deformity via generation or modification of the correction path for the bone but allow a specified bone deformity to remain in the final corrected stated. In some examples, disabling over/under correction may result in the correction logic circuitry automatically generating additional stages, waypoints, or code blocks to a correction path modified by the user so that only the specified remaining deformity will remain at the end of the prescription.

FIG. 2F-G illustrate examples of a user interface 2500 or 2600 for the correction logic circuitry to obtain input data about the location of the bone fixator hardware relative to the bone segments. In some examples, a point on the bone fixator is described relative to a point on the bone segments.

FIG. 2F illustrates an example of a user interface 2500 of the correction logic circuitry to facilitate graphical data entry of information about the bone fixator such as an external fixator. The correction logic circuitry may use image analysis presented in the user interface 2500 to graphically identify location the bone fixator hardware in the AP view 2510, the Lateral view 2520, and the Axial view 2530 via medical images (e.g., AP view and LAT view radiographs) or data inputs. In some examples, the correction logic circuitry may calculate the bone fixator location data automatically from the medical images via edge detection algorithms, radiographic markers, and the like.

FIG. 2G illustrates an example of a user interface 2600 of the correction logic circuitry that offers manual data entry of the hardware parameters as an alternative to automated data entry of hardware parameters based on image analysis of the bone fixator via the radiological images. In some examples, the user interface 2600 provides a user interface element (not shown) such as a manual mode button and an x-ray mode button to select the method of entry of the hardware parameter input data. In the present example, the manual mode button of the user interface element may be selected to select the user interface 2600 rather than a user interface 2500 as shown in FIG. 2F.

In some examples a single reference hardware component such as a ring is described relative to the bone segments. In the example of 2600, the mounting parameters are manually defined in the AP view 2612, Lateral View 2614, and Axial View 2616. The locations of additional hardware components of the bone fixator may be automatically defined by the means of connection to the referenced hardware component (e.g., the location of a second ring may be defined by the struts connecting the second ring to a reference ring). In some examples the connection constraints of specific hardware components are defined when they selected by the user via a data structure such as a library. In some examples, the user may directly input the location of all relevant hardware components into the correction logic circuitry.

FIG. 2H illustrates an example of a user interface 2600 of the correction logic circuitry that offers entry of rate limiting parameters for a prescription including a maximum safe distraction rate in millimeters per day and a maximum angulation (rotation) rate in degrees per day. In some examples, the user interface 2600 may include an option to “Apply Axial Translation First”. The option to “Apply Axial Translation First” may generate a first stage of the correction path to perform axial translation prior to performance of the AP angulation, LAT angulation, AP translation, LAT translation, and Axial angulation (rotation). In some examples, the user interface 2600 may also include a duration override option, “Override Duration” in number of days to allow the user to force a correction to end upon the input number of days.

In the present example, the user interface 2600 includes two options for automatic generation of the correction path including a first correction path optimized for the anatomy of the deformity correction and a second correction path for optimized for the struts of the bone fixator. Note that in other examples, the user interface 2600 may present additional automated correction path options and note that, in some examples, each option may include parameters of the correction path option such as the maximum translation rate implemented to generate the correction path, the maximum angulation rate implemented to generate the correction path, the number of strut change-outs implemented to generate the correction path, and the duration of the correction path.

FIG. 2I illustrates an example of a prescription with daily adjustments. Note that, in the presented example, the prescription begins with a first stage to correct axial translation that ends after the adjustment scheduled for the 16^{th} day of the prescription. Some examples with multiple steps of correction may indicate when each stage of correction begins and ends.

Each adjustment, which coincides with each day of the prescription in the present example, describes the adjustments for each of the struts. In the present example, the prescription presents the strut adjustments as the length of the strut for each of the six struts of the bone fixator. In other examples, the prescription may describe the adjustments to the length of the struts in addition to or in lieu of the length of the struts. For instance, strut 1 includes a strut length at day 0 of 180.00 mm and a strut length at day 1 of 181.00 mm. Thus, the length of the strut 1 is increased by 1 mm as part of the adjustment on day 1. The prescription may show the adjustment of 1 mm for day 1, 1 revolution of strut 1, 1 click of strut 1, or the like in addition to the overall length of the strut 1 or as an alternative to showing the overall length of the strut 1.

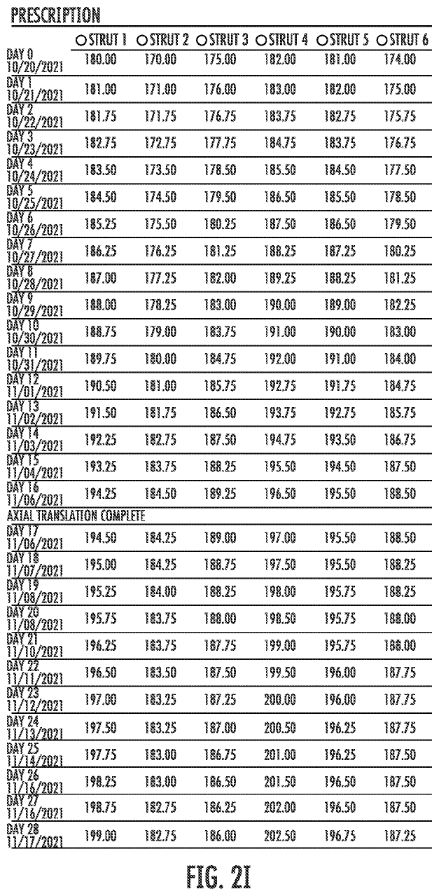

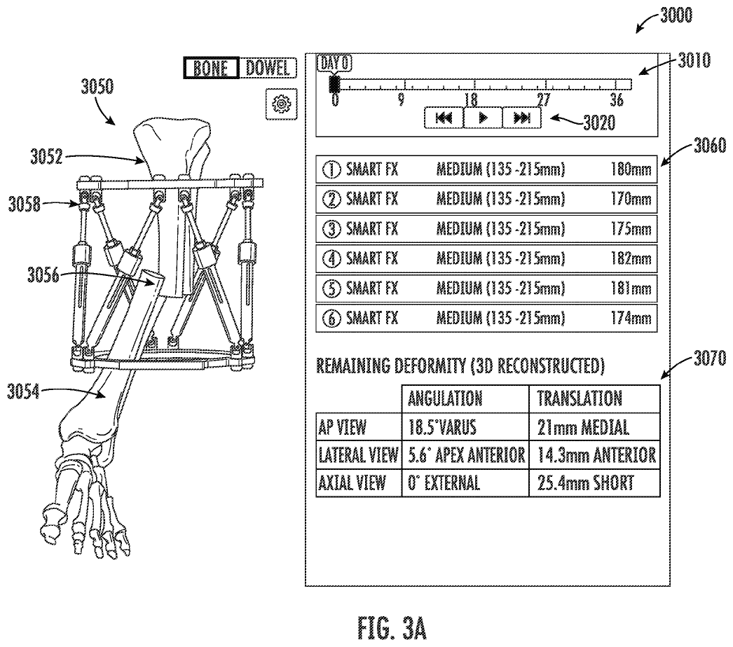

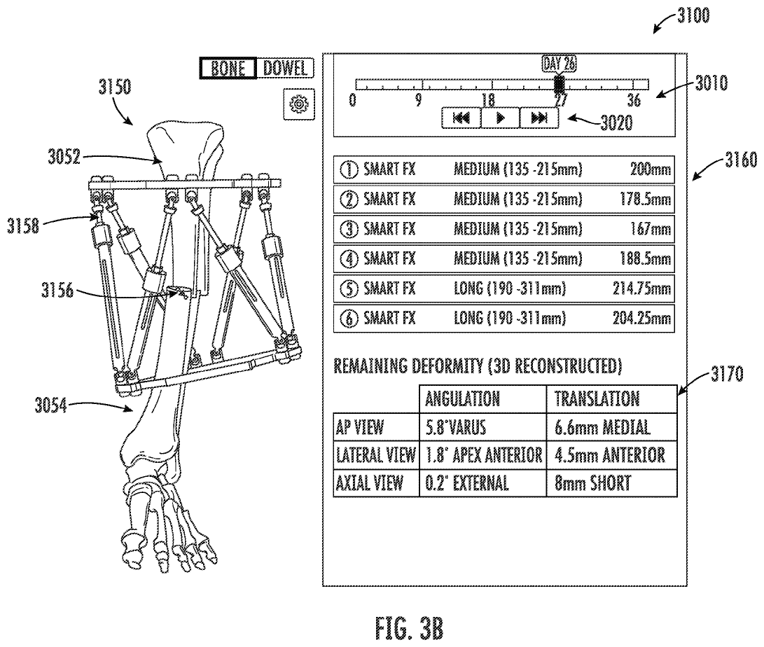

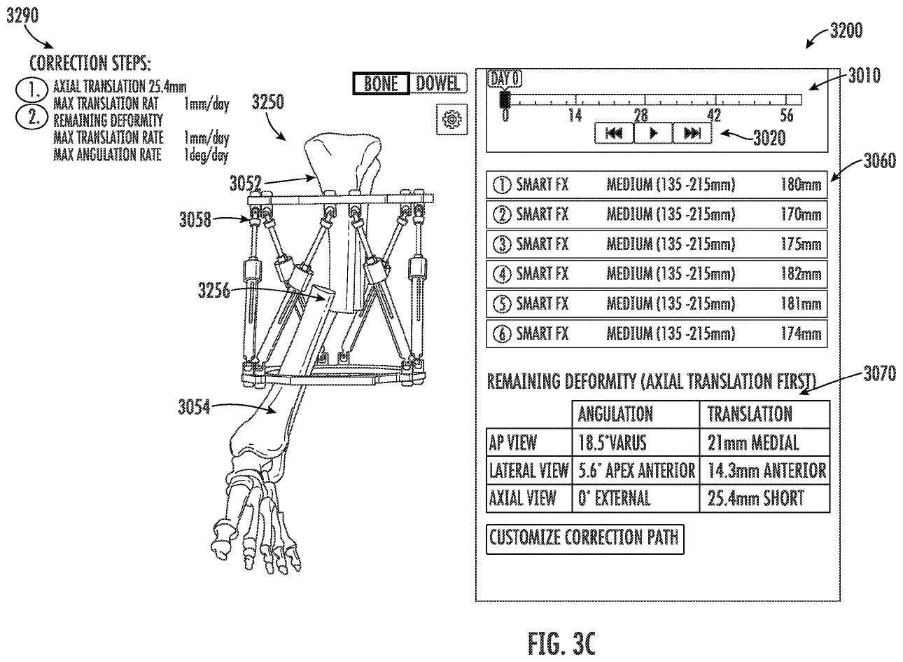

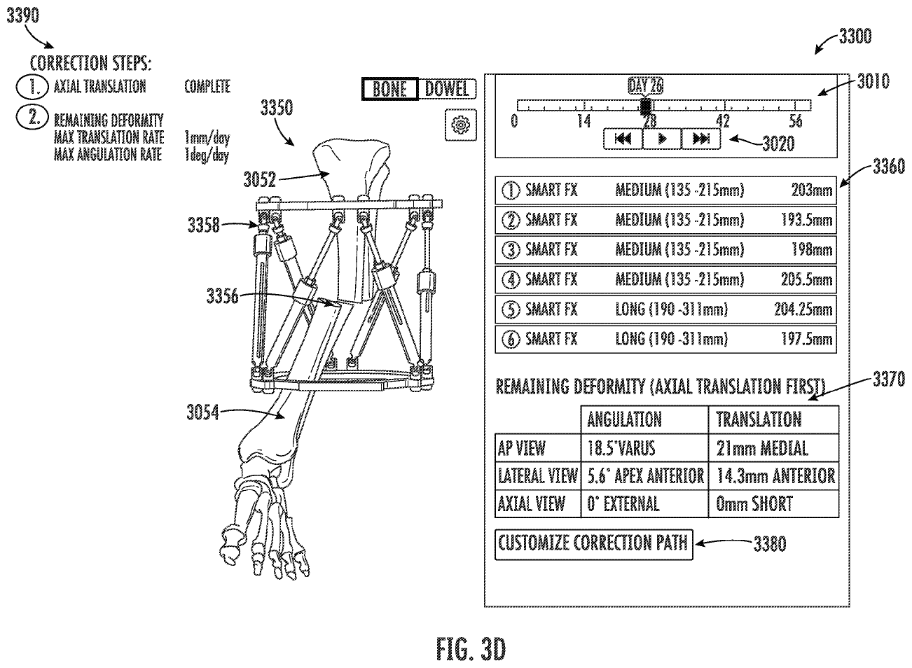

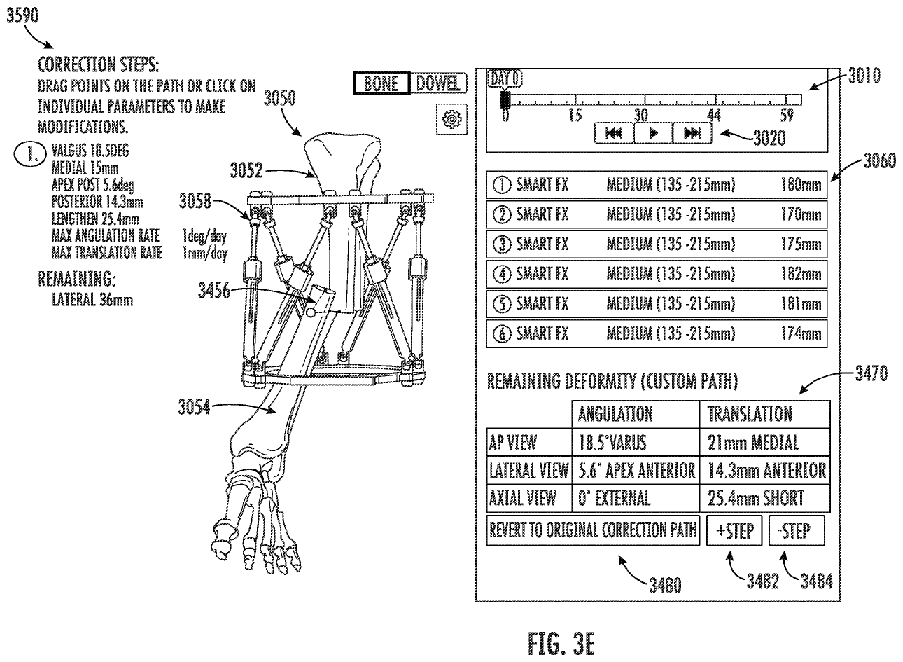

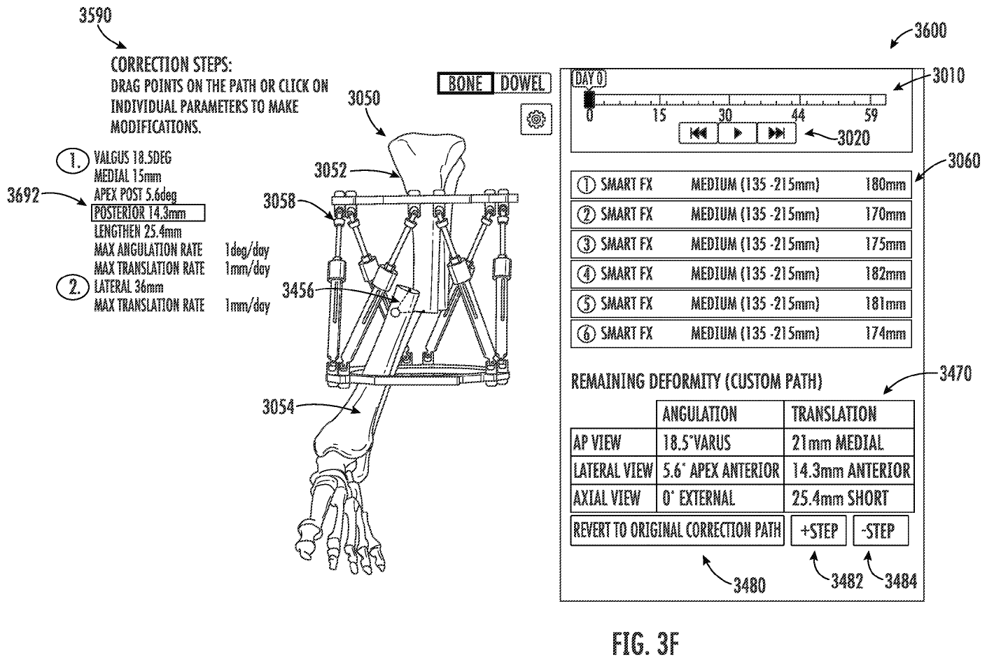

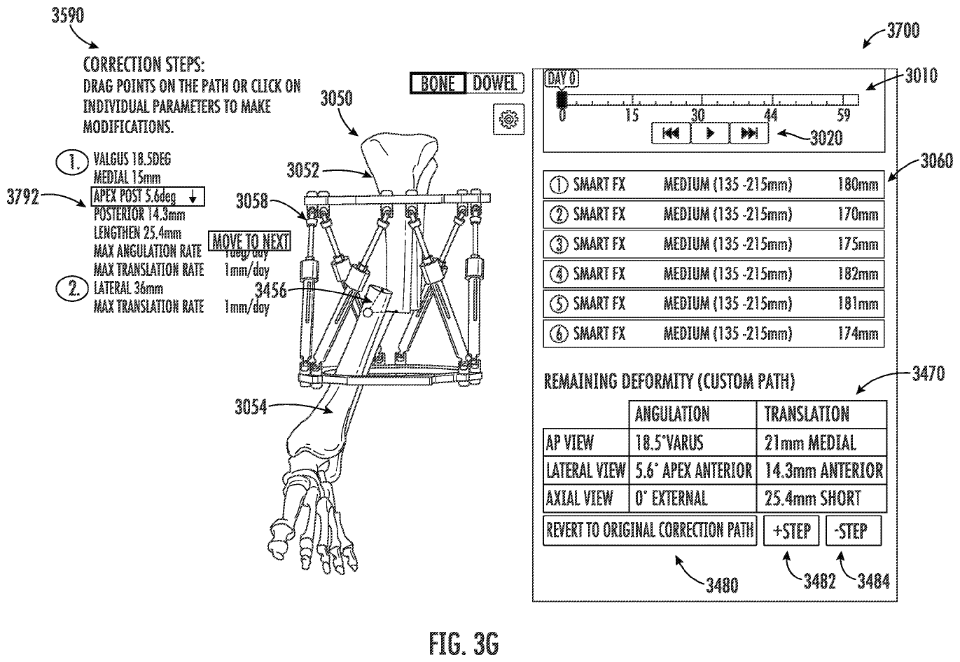

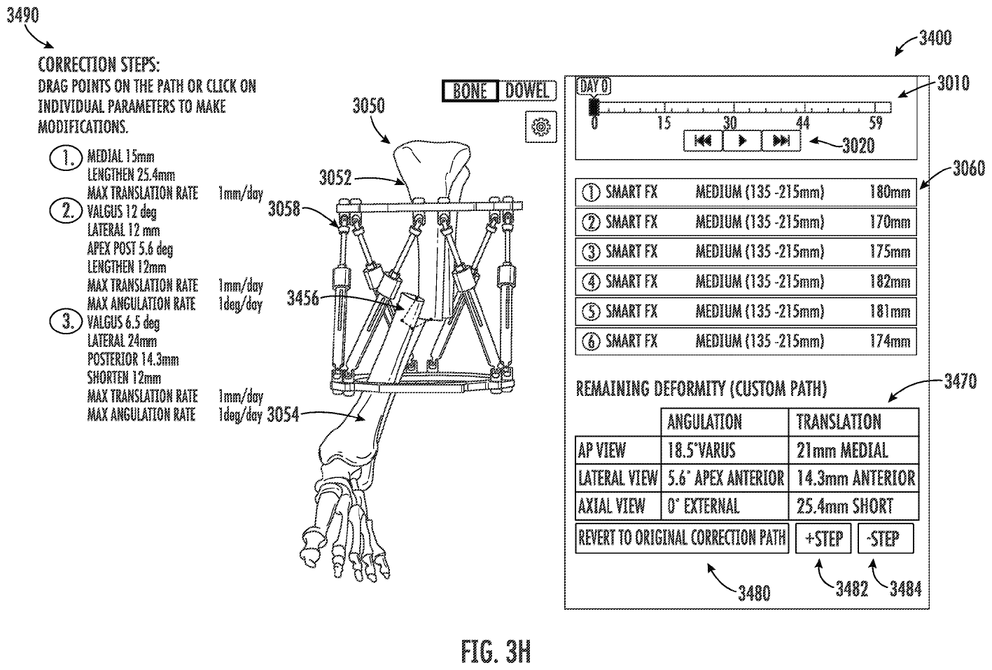

FIG. 3A-B illustrates an example of a user interface 3000 of correction logic circuitry for displaying a treatment plan or prescription with only one correction stage. The user interface 3000 may include a user interface element 3010 to select a day within the prescription. The user interface 3000 may also include a user interface element 3020 to play, rewind, and fast forward the prescription to illustrate the correction path of the moving bone segment 3054, show in the strut lengths 3060 for the displayed day of the prescription, show the minimum and maximum strut lengths for the installed struts, and show the remaining deformity 3070 after the daily adjustment for the displayed day of the prescription.

FIG. 3A illustrates an example in which, the user interface 3000 displays a graphical representation of a bone 3050 shows the position of the fixed bone segment 3052 at Day 0, the position of the moving bone segment 3054 at Day 0, the state of the bone fixator 3058 at Day 0, and the correction path 3056.

In some examples, the bone fixator 3058 will move in the graphical representation 3050 such that the moving bone segment 3054 will follow the correction path 3056 as the prescription advances. FIG. 3B illustrates the user interface 3100 for the prescription shown in FIG. 3A advanced to Day 26 of the prescription. The user interface element 3010, strut settings 3160, remaining deformity 3170, and graphical representation 3150 have all updated to represent the state of the bone fixator 3158 and moving bone segment 3154 after the prescribed adjustments for Day 26 of the prescription. The correction paths 3156 and 3056 are identical, but 3156 has advanced such that the previous 25 days are not visible. In some examples, the entire correction path may be displayed throughout the prescription.

In some examples with impingement analysis the user interface (3000 and 3100) may display a warning or message if impingement is likely to occur with the current correction path of the prescription. In other words, detected collisions may be communicated to the user via color changes, dialog boxes, onscreen warnings, and the like. The correction logic circuitry tracks the position and end edge geometry of the moving bone fragment, fixed bone fragment, bone fixator, input addition hardware components, and input transosseous elements throughout the prescription.

FIG. 3C-D illustrates an example of a user interface 3200 of correction logic circuitry for creating a treatment plan or prescription that corrects axial translation first. The user interface 3200 is the user interface 3000 in FIG. 3A except that user has selected to correct axial translation first.

FIG. 3C illustrates the graphical representation of a bone 3050 shows the state of the deformity on Day 0 and is a 3D image so the user may change the perspective by clicking and dragging points on or near the graphical representation of a bone 3050 in the direction of the desired perspective view. In many examples, the correction logic circuitry may also allow the user to zoom in on the image by double clicking on the image or via another user element for zoom that is not shown.

The legend 3290 shows the correction steps created by the user on the correction path 3256. Waypoints are created at the initial location of the moving bone segment, the location of the moving bone segment after the axial translation deformity, and after the remaining deformity is corrected following the final adjustment of the prescription. The correction logic circuitry calculates the prescription required to cause the moving fragment to follow the created correction path according to the rate limiting inputs. The first correction step shows the correction path the correction of the axial translation deformity, which will occur over the first 25 days of the prescription. The second correction step, which is automatically generated by the correction logic circuitry, solves the remaining deformity from Day 26 to Day 57 of the prescription. In some examples each correction stage will follow the same rate limiting inputs which define the duration of each correction step. In other examples each correction stage may utilize independent rate limits.

FIG. 3D illustrates an example of a user interface 3300 of correction logic circuitry for a treatment plan or prescription correcting the remaining deformity after the axial translation first correction stage has been completed. The user interface 3300 is the user interface 3000 in FIG. 3C except that user has advanced the day to Day 26 so the strut lengths 3360 shows the state (lengths and sizes) of the struts at Day 26. The remaining deformity 3370 shows the remaining deformity after the adjustment made on Day 26 and the graphical illustration of the bone 3350 shows the fixed bone segment 3052, the state of the bone fixator 3358, and the state of the moving bone segment 3054 after the adjustment made on Day 26 of the prescription.

The legend 3390 shows correction step (stage) for Axial translation is complete as well as the maximum rates for translation and angulation as 1 mm/day and 1 degree/day, respectively, for the remaining deformity correction. Furthermore, the user interface 3300 may show an arrow 3356 indicative of the correction path between Day 26 and Day 57 on the graphical illustration of the bone 3350.

In other examples, the user may select to correct a different deformity parameter component(s) first. For example, the user may select to correct AP Translation First or a combination of AP Translation and Axial Translation First. For AP Translation First the correction logic circuitry would automatically create waypoints at the initial position of the moving bone segment, at the position of the bone segment after the AP Translation deformity is corrected, and after the remaining deformity is corrected.

In some examples over/under correction may be enabled. If the example shown in FIG. 3C-D was processed the correction logic circuitry with an over correction 3 mm long and 1 degree varus selected, then the correction logic circuitry would automatically create waypoints at the initial location of the moving bone segment, the location of the bone segment after the Axial Translation deformity is corrected, and after the remaining deformity is corrected except the bone segment is 3 mm long and 1 degree varus of the fixed bone segment. In some examples, the correction logic circuitry may automatically create waypoints when over correction is enabled at the initial location of the moving bone segment, the location of the bone segment after the Axial Translation deformity is corrected, the location of the moving bone segment after the remaining deformity is corrected, and the location of the moving bone segment when the over correction is applied.

FIG. 3E-I illustrate examples of user interfaces of correction logic circuitry for customizing the correction path of a treatment plan or prescription. The user interfaces are similar to the user interface 3000 in FIG. 3C but begin with the deformity and prescription from FIG. 3A, which had a single correction stage.

FIG. 3E illustrates the user interface 3500 after the user clicked on the user interface element “Customize Correction Path” 3280, as shown in FIG. 3D, for DAY 0 and has begun to graphically drag one of the adjustment points of the correction path 3456 to a new location but has not applied the change. In some examples the correction logic circuitry may calculate and display the remaining deformity and prescription in real time that will result from the modification to the correction path. Other examples may display portions of the resulting data, such as the deformity change but not calculate the full prescription until the change is fully applied by the user.

After the user selects the user interface element “Customize Correction Path” 3280, in the present example, the user interface element 3280 changes to a user interface element “Revert to Original Correction Path” 3480 and two additional user interface elements appear including “+Step” 3482 and “−Step” 3484. With the user interface elements “+Step” 3482 and “−Step” 3484, the user can interact with the correction logic circuitry to add or delete waypoints to the correction path starting at the currently selected point in time. Each of the new waypoints creates a corresponding step in the “Correction Steps” legend 3490 and each step is a different stage in the correction path of the prescription. In some examples, an adjustment point of the correction path 3456 may be manipulated and then confirmed by the user by clicking interface elements “+Step” 3482.

FIG. 3F illustrates an example of a user interface 3600 of correction logic circuitry for customizing the correction path of a treatment plan or prescription. The user interface 3600 is the user interface 3500 in FIG. 3E at Day 0 but in this example, the user has created the second stage. Furthermore, the legend 3590 shows a highlighted correction path parameter 3692, Posterior 14.3 mm, that the user is editing with keystrokes. In other words, the user is typing a new value for Posterior with a keyboard of the, e.g., HCP device 140. The max angulation rate and max translation rate may be similarly adjusted. In some examples, when the keystrokes result in a deformity value other than the originally measured the deformity the correction logic circuitry will automatically store the difference as remaining deformity. The correction logic circuitry will automatically adjust the prescription to solve the remaining deformity and either incorporate the adjustments into a subsequent existing correction stage (e.g., the second correction stage) or create a new correction stage to solve the remaining deformity.

FIG. 3G illustrates an example of a user interface 3700 of correction logic circuitry for customizing the correction path of a treatment plan or prescription. The user interface 3700 is the similar to the user interface 3600 in FIG. 3F at Day 0 but in this example, the legend 3590 shows a highlighted correction path parameter 3792, Apex Post 5.6 degrees, that the user is editing with keystrokes via a keyboard of the, e.g., HCP device 140. In addition to editing the magnitude of the deformity being corrected, the user may elect to move the correction of a deformity parameter to a different step. In the present example the user may move the correction of 5.6 degrees Apex Anterior to the second correction stage. In some examples users may also move the correction of reordered deformity parameter to a previous correction stage (e.g., from 2 to 1). In all situations, the correction logic circuitry will automatically adjust the prescription of each stage to solve the updated correction path.

FIG. 3H illustrates an example of a user interface 3400 with three correction steps. The correction path 3456 depicts each correction step in a different format (e.g., color, line font, and the like). The correction steps 3490 are formatted to match to correction path. The first step corrects the deformity parameter components of Medial 15 mm and Lengthens 25.4 mm with a maximum translation rate of 1 mm per day. The second stage corrects the deformity parameter components of Valgus 12 degrees, Lateral 12 mm, Apex Posterior 5.6 degrees, and Lengthens 12 mm with a maximum translation rate of 1 mm per day and a maximum angulation rate of 1 degree per day. The third stage corrects the deformity parameters components of Valgus 6.5 degrees, Lateral 24 mm, Posterior 14.3 mm, and shorten 12 mm with a maximum translation rate of 1 mm per day and a maximum angulation rate of 1 degree per day to fully correct the deformity.

Note that the correction steps in the legend 3490 may be edited graphically by clicking on and dragging waypoints or adding or deleting waypoints via the user interface elements 3482 and 3484. In many examples, the user may, alternatively, select the numbers that the user intends to edit in the legend 3490 and edit the numbers with keystrokes via, e.g., a keyboard.

The remaining deformity 3470 shows the remaining deformity after the adjustment made at the current point in time, which is Day 0 in this example, and the graphical illustration of the bone 3050 shows the fixed bone segment 3052, the state of the bone fixator 3058, and the state of the moving bone segment 3054 after the adjustment made on Day 0 of the prescription. The arrow 3456 shows the customization of the correction path by the user by adding the new waypoints as correction steps or stages as described in the legend 3490.

FIG. 3I illustrates an example of a user interface 3800 of correction logic circuitry for customizing the correction path of a treatment plan or prescription. The user interface 3800 is the similar to the user interface 3800 in FIG. 3G at Day 0 but in this example, the end point of the prescription has been moved to cause an over correction. The legend 3890 shows an indication that the shorten parameter in the third stage is 19 mm, which is “(7 over)” to originally measured deformity. In other words, the shorten parameter is 7 mm over corrected, which means that the user has compressed the moving bone segment into the fixed bone segment by 7 mm. In some examples, this overcorrection is accomplished by graphically repositioning the final location of the moving bone segment in the correction path 3456.

In some examples, the correction logic circuitry may limit the correction path based on hardware constraints such that modifications stay within the correction paths that are possible for the hardware to follow. For example, the hardware of a bone fixator may be limited (hardware constraint) to minimum adjustments of 1 mm increments. The correction logic circuitry may limit the user to paths that are achievable with given hardware constraints.

In some examples, the correction logic circuitry may interact with a user to manipulate the correction path in a 3D display such as a representative model such as a model of a bone or a dowel. Some examples may also include overlaying the manipulatable correction path on statistical models, 3D medical images such as MRI, CT, etc., or overlaying 2D radiographic images on 3D models.

In some examples, the correction logic circuitry may manipulate the correction path in 2D on two planes and project the correction path in 3D. The correction logic circuitry may then calculate the closest 3D projection of the curves that on the two planes to generate one 3D curve. The two planes may include medical images of a deformity such as AP and Lateral radiographs. In some examples, the correction logic circuitry may limit the selectable portions of the initial manipulatable curve according to hardware restrictions or constraints.

In many examples, the described correction path adjustment is viable with any fixators that utilize software and is not limited to a hexapod as shown in the figures. Furthermore, the correction logic circuitry may alter the rate of correction in mm/day or deg/day for the correction path at each waypoint or stage of the correction path.

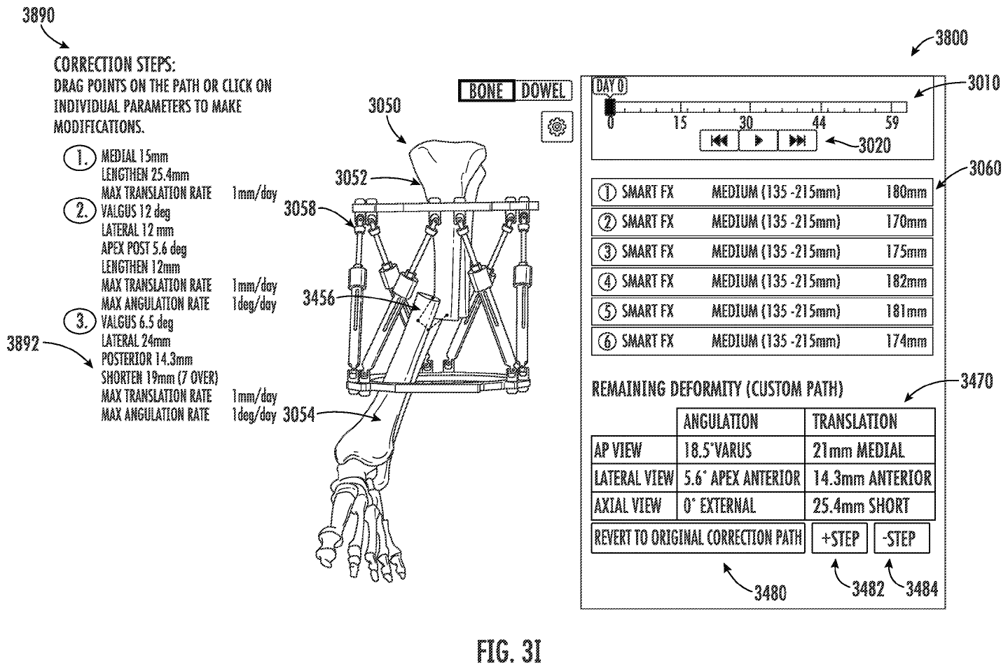

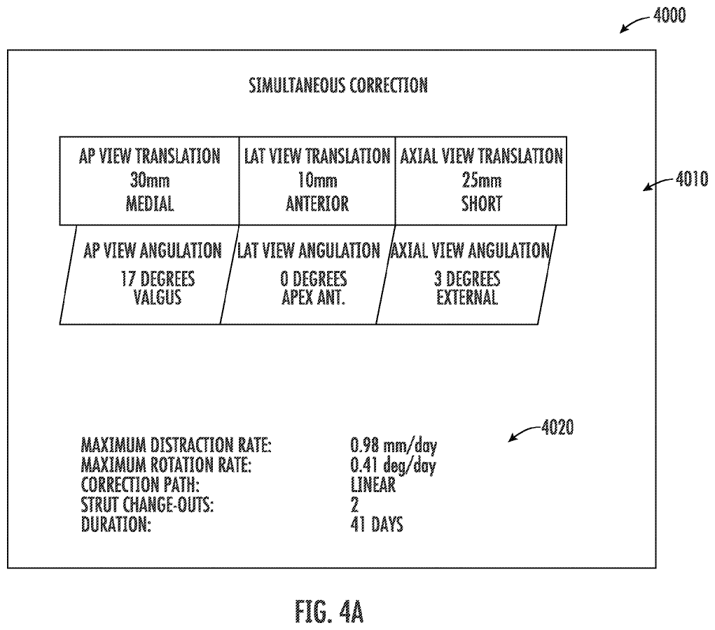

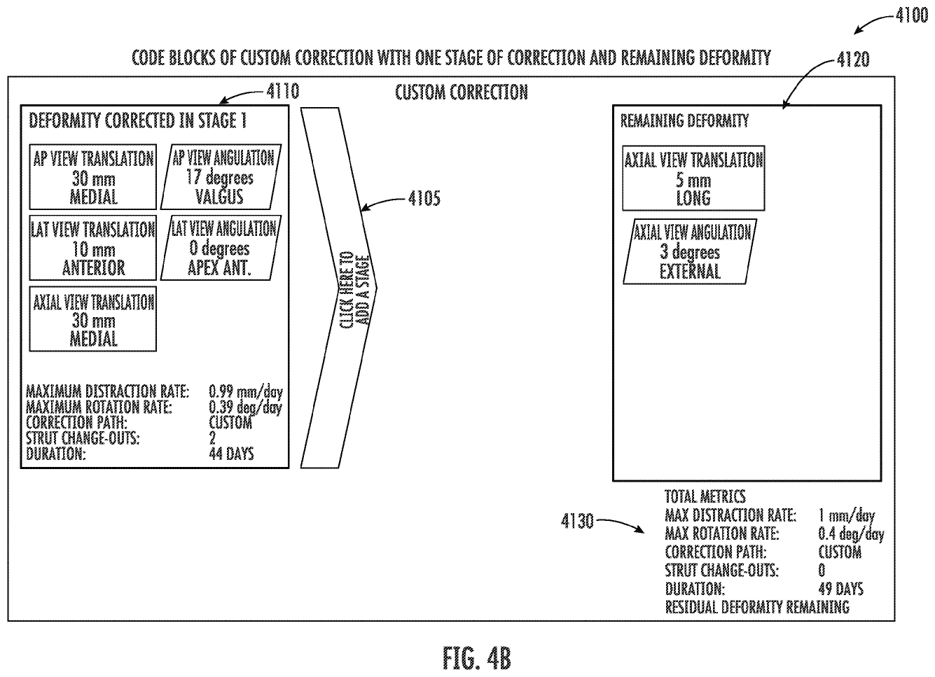

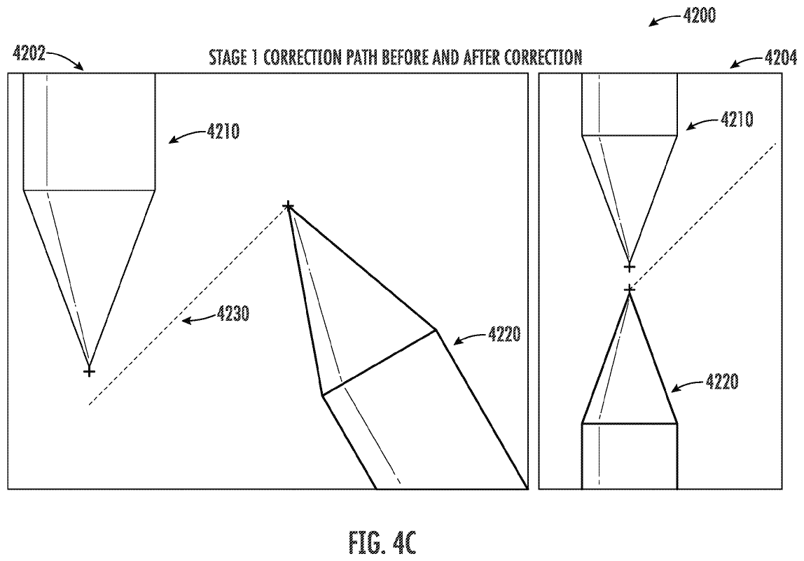

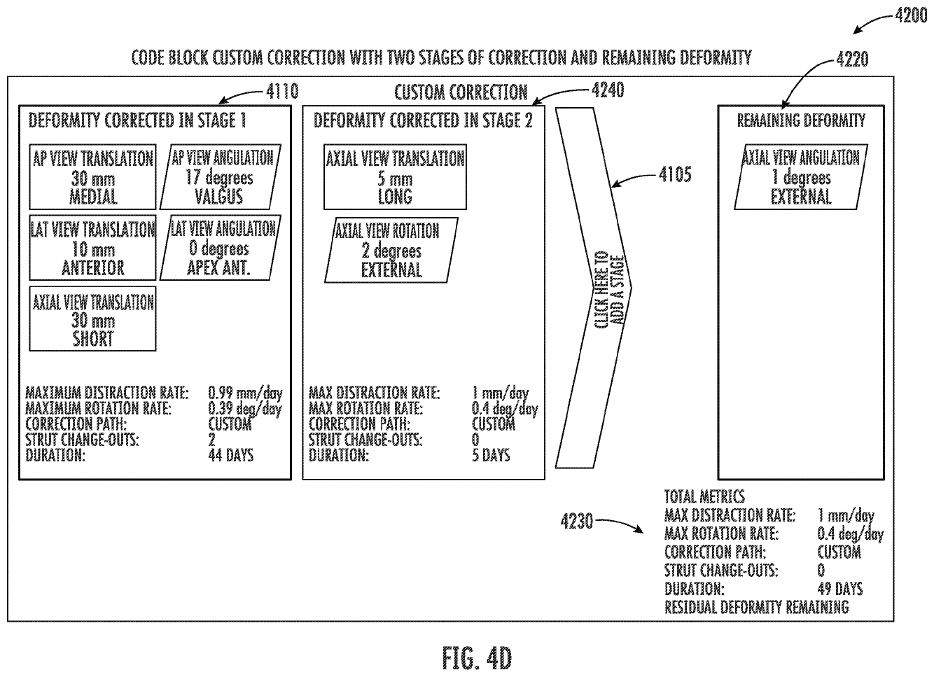

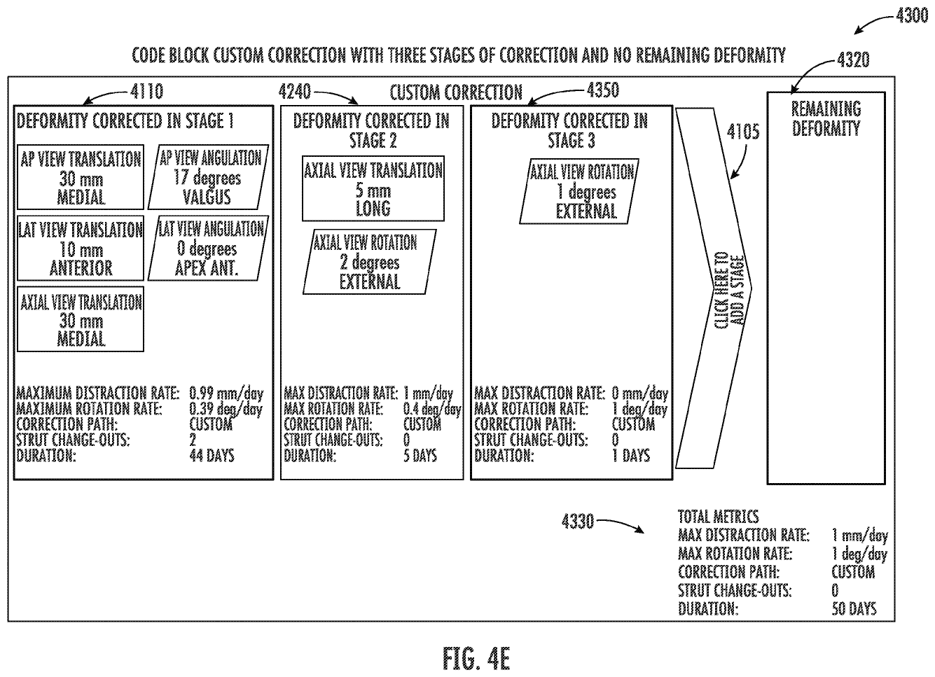

FIGS. 4A-E illustrate further examples of user interaction with a graphical interface having code blocks to adjust a correction path according to a prescription such. FIG. 4A illustrates the user interface 4000 of correction logic circuitry. The user interface 4000 includes six code blocks. Each code block 4010 represents a component of the deformity to be corrected during the correction path of the prescription such as an AP view translation, an AP view angulation, a LAT view translation, a LAT view angulation, axial view translation, and axial view angulation. The six deformity correction parameters 4010 may represent all the possible corrections for a correction path.

Users may adjust the correction path of a frame by breaking the path into one or more stages using a series of code blocks. In the current example, all deformity parameter components are to be solved simultaneously. Fixators utilizing software may require the deformity of the patient to be defined in all 6 degrees of freedom. Typically, deformity parameters take the form of AP View Angulation, AP View Translation, Lateral View Angulation, Lateral View Translation, Axial View Rotation (Angulation), and Axial View Translation. Once each deformity parameter is defined with a magnitude and direction, the correction logic circuitry may organize the deformity parameters into code blocks. By default, the correction logic circuitry may correct all deformity parameters according to the rate limits defined by the user as described in conjunction with FIGS. 2A-I.

For generation of the adjustments for the prescription, the correction logic circuitry may use the input data and user preferences such as the input data and user preferences described in conjunction with FIGS. 2A-H. In some examples, the correction logic circuitry may generate the adjustments at or near the maximum rate of translation and/or angulation. In some examples, the correction logic circuitry may generate the adjustments based on the number of days that the user set in user preferences for this prescription, that the user set in user preferences for prescriptions as a default, or that the user set in user preferences for this code block. In some examples, the correction logic circuitry may generate a linear correction path to correct the bone deformity based on the six deformity correction parameters 4010.

The legend 4020 may describe one or more of the maximum distraction rates the code blocks, the maximum rotation rate of the code block, the type of correction path as linear or non-linear, the number of hardware modifications such as the number of strut change-outs, and the duration of the prescription. In some examples each code block may have a rate limiting input rather than utilizing the same rate limits for an entire correction step or prescription. In some examples, the user may edit the values in the six deformity correction parameters 4010 with keystrokes or graphical interaction with a correction path on an image of the deformity, as discussed in conjunction with FIGS. 3A-3I, and, in some examples, may include a value of zero if the deformity correction parameter is not being implemented in the code block.

In some examples, the user interface 4000 may include one or more 2D or 3D images of the fixed and moving bone segments such as the images of the bones shown in FIGS. 3A-I. In some examples, the user interfaces described in conjunction with FIGS. 3A-3I are displayed along with the code blocks and the user may choose to create or modify a prescription via either or some combination of the user interfaces. In some examples, edits to the correction path on a graphical user interface such as the user interfaces described in FIGS. 3A-I may be automatically represented in the code block(s) of the user interface 4000 and vice versa. In further examples, the user may interact with the correction logic circuitry to switch between the code block user interface and the user interfaces described in conjunction with FIGS. 3A-3I.

FIG. 4B illustrates the user interface 4100 of correction logic circuitry. The user interface 4100 includes a first stage of correction 4110 and remaining deformity code blocks 4120. In the present example, the user created or modified the first stage of code blocks 4110 to include part of the deformity correction for a prescription. The remaining deformity code blocks 4120 may be automatically generated by the correction logic circuitry and may include the remaining deformity that the user indicated would correct the deformity in the input data. The user may generate one or more stages of code blocks to reduce or eliminate the remaining deformity in shown in the remaining deformity code blocks 4120 by interacting (e.g., clicking on) with the user interface element 4105.

In the present example, the user may elect to customize the correction path by generating or rearranging the code blocks into stages and arranging the stages into a desired order to change the correction path. When a code block is adjusted such that the moving bone segment has not reached its final corrected state, in some examples, the correction logic circuitry may generate a new code block for the remaining required movements. Thereafter, the user may adjust any combination of translation and angulation code blocks. In some examples, formatting may indicate to a user if a code block fully corrects a deformity parameter component, under corrects the deformity parameter component, or over corrects the deformity parameter component. The formatting may be in the form of colors of text or numbers, highlighting of text or numbers, additional text or numbers to show the magnitude of over or under corrections, and/or the like. In some examples, default or user specified preferences may establish text, text colors, and/or highlight colors that indicate whether the adjustments over correct, under correct, or completely correct deformity parameters.

In the present example, the user may have elected to distract the bone beyond what was needed for correction but elected not to correct Axial View Rotation (Angulation) in Stage 1 of the correction. The correction logic circuitry may calculate the new correction path and the resulting prescription according to the maximum distraction rate, rotation rate, and duration for the stage and the total correction. The change to Axial View Translation magnitude and direction left the moving bone segment 5 mm distal of the reference bone segment so the correction logic circuitry generated a new unapplied code block, the remaining deformity code block area 4120. In some examples, the remaining deformity code blocks are automatically added to a new stage of correction rather than being stored unapplied.

In some examples, the user interface 4100 may also include a legend 4130 that includes the total metrics of the current correction path of the prescription. The legend 4130 includes a maximum distraction rate of 1 mm per day, a maximum rotation rate of 0.4 degrees per day, an indication that the current correction path is custom, an indication of hardware modifications needed to perform the deformity correction of the current correction path, a duration of 49 days for completing the adjustments of the correction path in accordance with user-imposed limitations in user preferences, and an indication that there is a residual deformity remaining. In some examples, the indication about the correction path may include an indication that the path is original or an automatically generated path by the correction logic circuitry.

FIG. 4C illustrates a user interface the result of a correction path in FIG. 4B with remaining deformity components. The fixed bone segment 4210 and a moving bone segment 4220 are depicted on the initial day of the resulting prescription 4202 and after the final day of the resulting prescription 4204. The fixed bone segment 4210 remains stationary while the moving bone segment 4220 follows the correction path 4230. Note that the correction path 4230 includes an under correction such that a remaining deformity in the form of axial translation is uncorrected, which is the distance between the fixed bone segment 4210 and the moving bone segment 4220 in the after 4204 correction illustration. In some examples, the user may correct the remaining deformity in a subsequent stage or may leave the remaining deformity uncorrected in the final corrected state.

FIG. 4D illustrates the user interface 4200 of correction logic circuitry. The user interface 4200 includes the first stage correction of code blocks 4110 from FIG. 4B, a second stage correction of code blocks 4240, and a remaining deformity block 4220. In the present example, the user created or modified the first stage code blocks 4110 to include part of the deformity correction for a prescription. The user creates a second stage 4240 to correct the remaining Axial View Translation deformity resulting from excess lengthening in the first stage 4110 and resolve 2 degrees of Axial View Rotation (Angulation). The Final degree of Axial View Rotation (Angulation) is unapplied and stored at remaining deformity.

FIG. 4E illustrates the user interface 4300 solving the final degree of Axial View Rotation from FIG. 4D in the third correction stage 4350 so that the full deformity is corrected. The Remaining Deformity area 4320 is empty since no deformity parameter components remain unaccounted for.

In some examples, the correction logic circuitry may automatically update the legend 4230 in FIG. 4D after the creation of the second stage code blocks 4240 and update the legend 4330 in FIG. 4E after the creation of the third stage code blocks 4340. Note also that each code block includes a legend displaying metrics related to the correction paths associated with the corresponding code block. The metrics may also be further segmented to provide data for each individual code block.

In many examples, users may interact with the correction logic circuitry to choose how the use the remaining deformity code blocks and may choose to proceed without resolving the full bone deformity. Although not shown, the user could for example decide to add a fourth Stage to compress the fracture/osteotomy. An advantage of the custom correction paths according to some examples disclosed herein is that such examples may offer the user limitless combinations or solutions for deformity correction within a single prescription. For instance, in some examples, deformity parameters may be fully or partially corrected in any order while maintaining full control over the rate at which the deformity parameters are corrected. Deformity corrections that were previously performed in multiple adjustment schedules or prescriptions may now be combined into a one comprehensive adjustment schedule or prescription.

In some examples, the correction path defined by code blocks may be plotted in a 3D display or as two 2D projections of the 3D path. In some examples, the code blocks may function on their own or may be combined with the graphical modifiers such that the code blocks order and contents are generated according to the graphically modified path. In some examples, the generated code blocks may be further modified thus modifying the graphical display. In some examples, the rate of correction in mm/day or deg/day may be altered for each stage.

In some examples, the code blocks may record the deformity being corrected. In some examples, the code blocks may record the movement of the bone segment/frame (opposite of the deformity). For example, if the deformity was 30 degrees valgus, the code block for movement may record rotate 30 degrees varus or may record correct 30 degrees of valgus. In some examples, the code blocks may combine movement from one view or multiple views together.

As discuss herein, the correction logic circuitry may implement a user interface for code blocks to advantageously offer a user full customization of the correction path in an easy-to-use manner. In some examples, the remaining deformity may always be displayed so that users advantageously keep track of the final corrected state while customizing the correction path. In some examples, the correction logic circuitry may advantageously track the position of the moving bone segment after each adjustment to ensure that limiting inputs and hardware limits are followed.

In some examples, the users interact with the correction logic circuitry to advantageously perform as many movements, waypoints, and/or stages as they wish within one prescription rather than having to break it up into multiple prescriptions. For example, one prescription may distract, correct angulation, and then compress a deformity via the correction path.

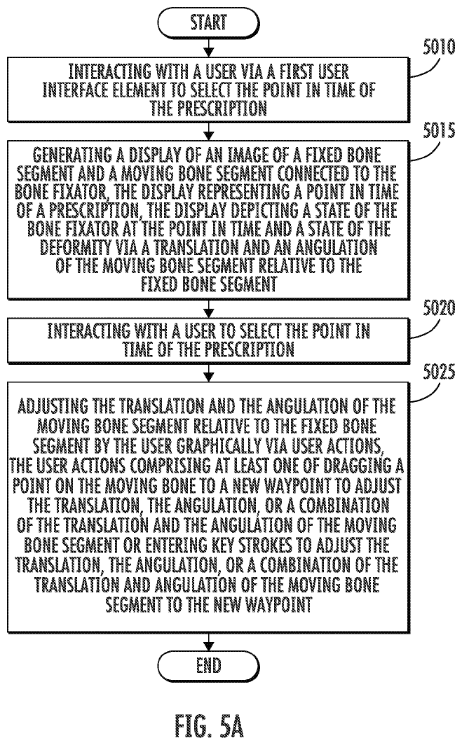

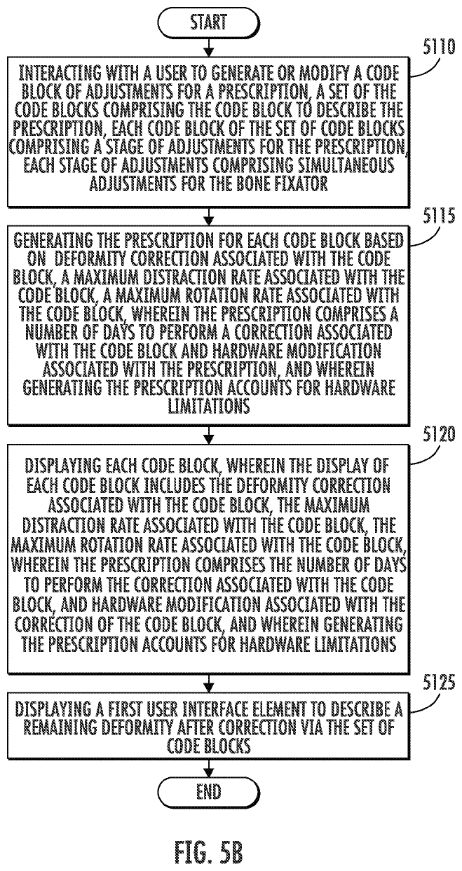

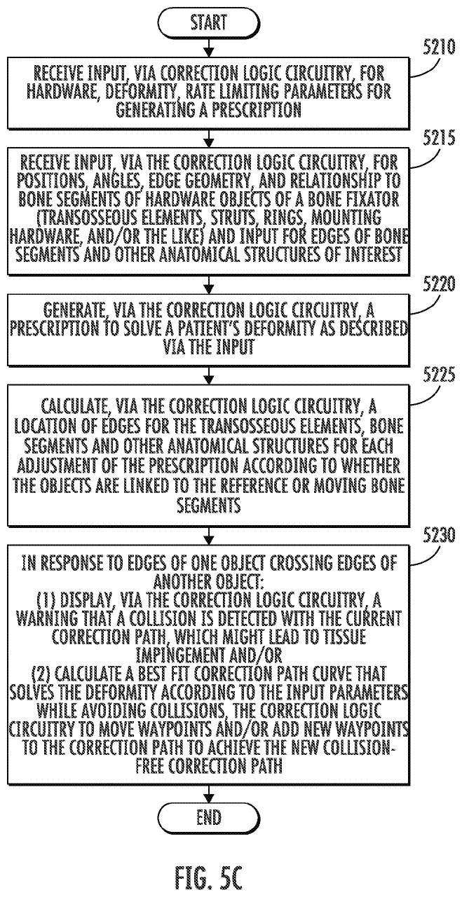

FIG. 5A depicts a flowchart 5000 of examples to modify the correction path of a prescription. The flowchart 5000 starts with interacting with a user via a first user interface element to select the point in time of the prescription (element 5010). For instance, a server such as the server 130 in FIG. 1A may include correction logic circuitry to transmit or identify two scaled radiographs or other 2D scaled images, or 3D imaging for a patient or to interact with a user of a computer such as the HCP device 140 in FIG. 1A. The user may identify a prescription determined for correction of the bone segments in the image(s) and either upload a file descriptive of the deformity and the fixator coupled with the bone segments of the bone or manually identify the postoperative deformity of the bone in the image(s) as well as the postoperative attachment of the fixator to the bone segments.

After identifying the imaging, the deformity, and the fixation of the fixator, the computer may generate a display of the image of a fixed bone segment and a moving bone segment interconnected with the bone fixator (element 5015). The display may represent a point in time of the prescription for the correction of the bone such as a prescription automatically generated based on the deformity of the bone. For instance, the prescription may include 28 days of adjustments such as one adjustment each day for 28 days. Each adjustment may involve a change to, e.g., the length of one or more struts of the fixator and/or replacement of one or more struts with alternative struts of a specified length. In such examples, the time before and the time after each adjustment or each day may represent a different point in time for the prescription.

In some examples, the display may not include the bone fixator. For example, the image may comprise an image of the bone segments to illustrate the bone deformity. In some examples, the image of bone deformity may comprise a bone model based on the user inputs, that illustrates the moving and fixed bone segments rather that displaying an actual image of the bone. In some examples, the image may include actual 2D or 3D images of the bone.

In some examples, the correction logic circuitry may present the automated adjustments to the user for approval along with the display of representations of the adjustments graphically on an image of the bone segments, in code blocks, with numerical corrections to the adjustments, and/or a combination thereof. In other examples, the correction logic circuitry may provide access to the corrected prescription and optionally illustrate the automated adjustments.

After generating a display of the image(s), the user may interact with the correction logic circuitry graphically or via keystrokes to select the point in time of the prescription and may also select a perspective view of the display (element 5020). The correction logic circuitry may include a user interface element such as a slide bar, pull down menu, counter, or the like to select one of the days (or adjustments) of the prescription to select the point in time of the prescription that the user wants to view. In some examples, the user may scroll through the days (or adjustments) of the prescription in a forward or a reverse order to watch the progress of the bone segment alignment throughout the prescription.

Thereafter, the correction logic circuitry may interact with a user to adjust the translation and the angulation of the moving bone segment relative to the fixed bone segment via user actions (element 5025). The user actions may include dragging a point on the moving bone to a new waypoint to adjust the translation, the angulation, or a combination of the translation and the angulation of the moving bone segment. In some examples, the user actions may include entering keystrokes to adjust the translation, the angulation, or a combination of the translation and angulation of the moving bone segment to the new waypoint.

In some examples, the correction logic circuitry may include a user interface element to describe the remaining deformity in response to identification of the new waypoint. For instance, the inclusion of the new waypoint may add one or more new adjustments to the prescription based on the maximum translation and/or rotation per day (or per adjustment period) and, as a result, change the number of days required to complete the prescription. On the other hand, the inclusion of a new waypoint that has a small adjustment to the current prescription may not add a new adjustment but may change a current adjustment and not change the number of days required to complete the prescription.

In many examples, the correction logic circuitry may generate a modified prescription accounting to record the changes made through interaction with the user. The modified prescription accounting may include modification of one or more translational components of the correction path or translational bone deformity, modification of one or more angular components of the correction path or angular bone deformity, or a combination thereof. In some examples, the modified prescription accounting may be recorded as a modified correction path in addition to or as an alternative to recording or storing the modified prescription accounting as one of more components of the translational deformity and/or one of more components of the angular deformity.

In some examples, the modified prescription accounting may group, through user interaction or autonomously, translational and angular corrections of the moving bone segment into one or more correction stages. The translational and angular corrections of the moving bone segment within the same correction stage may occur simultaneously according to associated maximum correction rates. In some examples, any partial remaining deformity components for the bone that are not assigned to the one or more correction stages are excluded, changing the final corrected state and allowing for under correction or over correction of one or more components of the bone deformity.

FIG. 5B depicts a flowchart 5100 of an example for a code block interface to adjust a correction path for a bone fixator. Note that some examples include the graphical interface discussed in FIGS. 3A-3I, FIGS. 4A-4E, and/or FIG. 5A as well as the code block interface discussed in conjunction with FIG. 5B.

The flowchart 5100 may begin with interacting with a user to generate or modify a code block of adjustments for a prescription (element 5110). A prescription may include one or more code blocks and each code block may include the deformity parameters: AP view translation, AP view angulation, LAT view translation, LAT view angulation, axial view translation, and axial view angulation. While each code block may modify a deformity parameter component, the code blocks do not necessarily include changes to all the deformity parameters. The user may choose which of the deformity parameters to adjust in each code block.

In some examples, the code blocks may be grouped into a correction stage in the correction path of the prescription. In such examples, the adjustments to the deformity parameters in the code block are considered simultaneous. In other words, the correction logic circuitry may determine the number of days for a set of adjustments for each grouping of code block for the fixator based on limitations placed on the amount of change per day to each of the adjustments simultaneously. Many examples are also based on the number of days on the user preferences and avoidance of impingement by the components of the bone fixator and, in some examples, neurovascular structures, bone segments, soft tissue, and/or the like.

Each stage of adjustments may include simultaneous adjustments for the bone fixator and each of the different stages may include adjustments to perform in series by the bone fixator. Thus, the correction logic circuitry may determine the set of adjustments for each code block and add the adjustments from the code blocks together to determine the prescription. Similarly, the correction logic circuitry may determine the total number of days for each of the stages and the total number of days for the prescription may include a sum of the number of days for each of the correction stages.

After interacting with the user to generate or modify a correction stage, the correction logic circuitry may generate the prescription for each correction stage based on a deformity correction associated with the code blocks, a maximum distraction rate associated with the code blocks, a maximum rotation rate associated with the code blocks, and/or the like (element 5115). The prescription may include a number of days to perform a correction associated with the correction stage and hardware modification associated with the adjustments represented by the correction stage. Furthermore, generation of the prescription may account for hardware limitations of the bone fixator. For instance, some translation and/or rotation adjustments may require struts of the bone fixator to be changed out for longer struts or shorter struts and a user display element of the correction stage may include an indication of any hardware modification required to perform the adjustments described in the correction stage.

In some examples, calculating the prescription may involve calculation by the correction logic circuitry of one three-dimensional linear curve for adjustments of the correction path based on correction paths described in code blocks and/or correction paths drawn graphically on two or more 2D images of the bone segments.

In some examples, the correction logic circuitry may generate and record a modified prescription accounting that includes the modifications made to the original prescription and, in some examples, includes a modified prescription. In some examples, any remaining code blocks not assigned to the one or more correction stages are excluded from the modified prescription, allowing for under-correction and/or over-correction of one or more components of the bone deformity. For instance, the modified prescription accounting may include an under-correction or over-correction of translational components of the bone deformity and may include an under-correction or over-correction of angular components of the bone deformity. In such examples, the modified prescription may have a partial deformity remaining in the final corrected state of the bone after completion of the modified prescription.

Once the correction logic circuitry generates or updates the prescription, the correction logic circuitry may display each code block, wherein the display of each code block includes the deformity correction associated with the code block, the maximum distraction rate associated with the code block, the maximum rotation rate associated with the code block, and/or the like (element 5120). The prescription may include the number of days to perform the correction associated with the code block, and hardware modification associated with the correction of the code block. Furthermore, generation of the prescription may account for hardware limitations. For instance, the correction logic circuitry may include a set of hardware limitations for the specific bone fixator installed on the bone segments. The hardware limitations may include, for example, the minimum adjustment possible for a strut such as 0.5 mm, 1 mm, or 2 mm.

In many examples, the correction logic circuitry may also display a user interface element to describe a remaining deformity after correction via the set of code blocks (element 5115). For instance, the user may create or modify code blocks to perform a first stage of adjustments to the bone fixator. The adjustments may not completely correct the deformity of the bone segments and the user element may describe the remaining deformity to correct. In some situations, the user may decide not to completely correct the deformity so the user may review the user element to determine if further adjustments are required or if the remaining deformity is the final corrected state for the prescription. If the user intends to completely correct the deformity, the values in the user element for the remaining deformity may inform the user about the amount of deformity left to correct in one of the current code blocks or in one or more new code blocks to reach the final corrected state.

FIG. 5C illustrates the flowchart 5200 of an impingement analysis example. At element 5210, the correction logic circuitry may receive input from the user for the hardware, deformity, and rate limiting parameters or receive the input based on selection of one or more files by the user, for generating a prescription with the correction logic circuitry, as illustrated in FIG. 2A-I. In some examples, the hardware parameters may include mounting parameters, relating the location of the hardware components or objects of a bone fixator to the bone segments. In some examples, deformity parameters and rate limiting parameters may include additional anatomical parameters.

The correction logic circuitry may receive input from the user through interaction graphically, via key entry, or via selection of one or more files to upload. The input may include positions, angles, edge geometries, and relationships to the bone segments of hardware objects of the bone fixator (transosseous elements, struts, rings, mounting hardware, and/or the like) (element 5215). In some examples, the user may also input or upload, and the correction logic circuitry may receive input for, edges of the bone segments and any other anatomical features of interest.

The correction logic circuitry may generate the prescription to solve the patient's deformity (element 5220) and calculate the locations of the edges for objects such as all input transosseous elements, struts, rings, mounting hardware, bone segments, anatomical structures, and/or the like for each adjustment of the prescription (element 5225). Hardware objects may either remain stationary with the fixed bone segment or move relative to the moving bone segment.

If the edges of one object cross the edges of another object during one or more adjustments of the prescription, then the correction logic circuitry will take action (element 5230). In some examples, the correction logic circuitry may display a warning that a collision is detected with the current correction path that might lead to tissue impingement. In such examples, the user may then elect to modify the correction path via graphical methods or code blocks.

In other examples, the correction logic circuitry may calculate a best fit correction path curve that solves deformity according to the input parameters while avoiding collisions. In some examples, the correction logic circuitry may add new waypoints to the correction path, such as correction of axial translation first, to achieve the new collision-free correction path. In some examples, the correction logic circuitry may display a collision warning to the user and calculate a collision free best fit correction path curve.

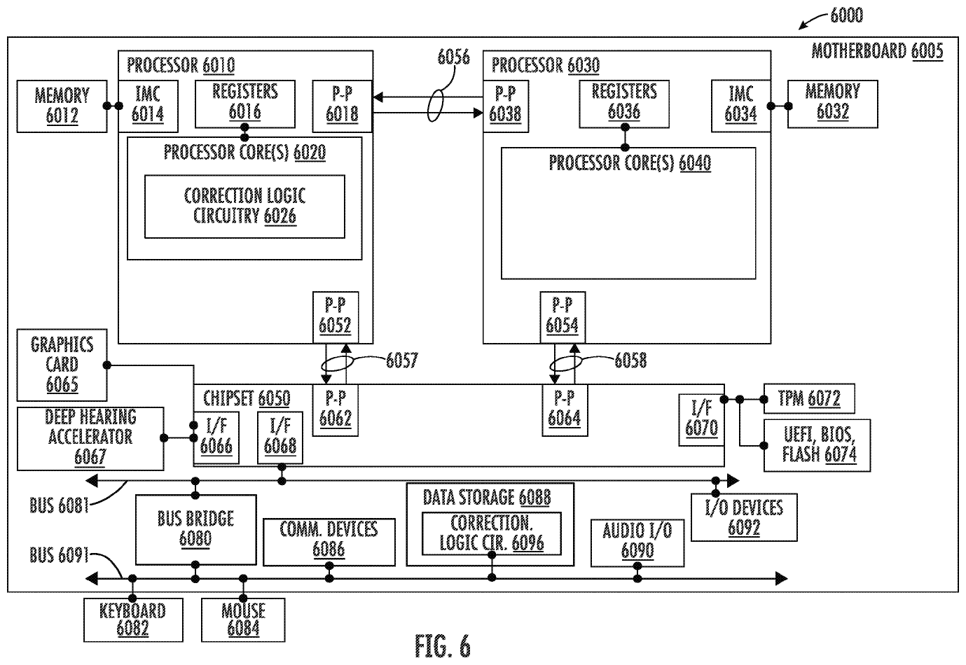

FIG. 6 illustrates an example of a system 6000. The system 6000 is a computer system with multiple processor cores such as a distributed computing system, supercomputer, high-performance computing system, computing cluster, mainframe computer, mini-computer, client-server system, personal computer (PC), workstation, server, portable computer, laptop computer, tablet computer, handheld device such as a personal digital assistant (PDA), or other device for processing, displaying, or transmitting information. Similar examples may include, e.g., entertainment devices such as a portable music player or a portable video player, a smart phone or other cellular phone, a telephone, a digital video camera, a digital still camera, an external storage device, or the like. Further examples implement larger scale server configurations. In other examples, the system 6000 may have a single processor with one core or more than one processor. Note that the term “processor” refers to a processor with a single core or a processor package with multiple processor cores.

As shown in FIG. 6, system 6000 includes a motherboard 6005 for mounting platform components. The motherboard 6005 is a point-to-point interconnect platform that includes a first processor 6010 and a second processor 6030 coupled via a point-to-point interconnect 6056 such as an Ultra Path Interconnect (UPI). In other examples, the system 6000 may be of another bus architecture, such as a multi-drop bus. Furthermore, each of processors 6010 and 6030 may be processor packages with multiple processor cores including processor core(s) 6020 and 6040, respectively. While the system 6000 is an example of a two-socket (2S) platform, other examples may include more than two sockets or one socket. For example, some examples may include a four-socket (4S) platform or an eight-socket (8S) platform. Each socket is a mount for a processor and may have a socket identifier. Note that the term platform refers to the motherboard with certain components mounted such as the processors 6010 and the chipset 6050. Some platforms may include additional components and some platforms may only include sockets to mount the processors and/or the chipset.

The first processor 6010 includes an integrated memory controller (IMC) 6014 and point-to-point (P-P) interconnects 6018 and 6052. Similarly, the second processor 6030 includes an IMC 6034 and P-P interconnects 6038 and 6054. The IMC's 6014 and 6034 couple the processors 6010 and 6030, respectively, to respective memories, a memory 6012 and a memory 6032. The memories 6012 and 6032 may be portions of the main memory (e.g., a dynamic random-access memory (DRAM)) for the platform such as double data rate type 3 (DDR3) or type 4 (DDR4) synchronous DRAM (SDRAM). In the present example, the memories 6012 and 6032 locally attach to the respective processors 6010 and 6030. In other examples, the main memory may couple with the processors via a bus and shared memory hub.

The processors 6010 and 6030 include caches coupled with each of the processor core(s) 6020 and 6040, respectively. In the present example, the processor core(s) 6020 of the processor 6010 include a correction logic circuitry 6026 such as the correction logic circuitry discussed in conjunction with FIGS. 1-5. The correction logic circuitry 6026 may represent circuitry configured to implement the functionality to adjust a correction path for bone fixator or bone segments connected to a bone fixator to generate a prescription of adjustments for a bone fixator to correct a bone deformity within the processor core(s) 6020 or may represent a combination of the circuitry within a processor and a medium to store all or part of the functionality of the comprehensive logic circuitry 6026 in memory such as cache, the memory 6012, buffers, registers, and/or the like. In several examples, the functionality of the correction logic circuitry 6026 resides in whole or in part as code in a memory such as the correction logic circuitry 6096 in the data storage unit 6088 attached to the processor 6010 via a chipset 6050 such as the correction logic circuitry discussed in FIGS. 1-5. The functionality of the correction logic circuitry 6026 may also reside in whole or in part in memory such as the memory 6012 and/or a cache of the processor. Furthermore, the functionality of the correction logic circuitry 6026 may also reside in whole or in part as circuitry within the processor 6010 and may perform operations, e.g., within registers or buffers such as the registers 6016 within the processor 6010, or within an instruction pipeline of the processor 6010.

In other examples, more than one of the processors 6010 and 6030 may include

functionality of the correction logic circuitry 6026 such as the processor 6030 and/or the processor within the deep learning accelerator 6067 coupled with the chipset 6050 via an interface (I/F) 6066. The I/F 6066 may be, for example, a Peripheral Component Interconnect-enhanced (PCI-e).

The first processor 6010 couples to a chipset 6050 via P-P interconnects 6052 and 6062 and the second processor 6030 couples to a chipset 6050 via P-P interconnects 6054 and 6064.

Direct Media Interfaces (DMIs) 6057 and 6058 may couple the P-P interconnects 6052 and 6062 and the P-P interconnects 6054 and 6064, respectively. The DMI may be a high-speed interconnect that facilitates, e.g., eight Giga Transfers per second (GT/s) such as DMI 3.0. In other examples, the processors 6010 and 6030 may interconnect via a bus.

The chipset 6050 may include a controller hub such as a platform controller hub (PCH). The chipset 6050 may include a system clock to perform clocking functions and include interfaces for an input/output (I/O) bus such as a universal serial bus (USB), peripheral component interconnects (PCIs), serial peripheral interconnects (SPIs), integrated interconnects (I2Cs), and the like, to facilitate connection of peripheral devices on the platform. In other examples, the chipset 6050 may include more than one controller hub such as a chipset with a memory controller hub, a graphics controller hub, and an I/O controller hub.

In the present example, the chipset 6050 couples with a trusted platform module (TPM) 6072 and the unified extensible firmware interface (UEFI), BIOS, Flash component 6074 via an interface (I/F) 6070. The TPM 6072 is a dedicated microcontroller designed to secure hardware by integrating cryptographic keys into devices. The UEFI, BIOS, Flash component 6074 may provide pre-boot code.

Furthermore, chipset 6050 includes an I/F 6066 to couple chipset 6050 with a high-performance graphics engine, graphics card 6065. In other examples, the system 6000 may include a flexible display interface (FDI) between the processors 6010 and 6030 and the chipset 6050. The FDI interconnects a graphics processor core in a processor with the chipset 6050.

Various I/O devices 6092 couple to the bus 6081, along with a bus bridge 6080 which couples the bus 6081 to a second bus 6091 and an I/F 6068 that connects the bus 6081 with the chipset 6050. In some examples, the second bus 6091 may be a low pin count (LPC) bus. Various devices may couple to the second bus 6091 including, for example, a keyboard 6082, a mouse 6084, communication devices 6086 and a data storage unit 6088 that may store code such as the correction logic circuitry 6096. Furthermore, an audio I/O 6090 may couple to second bus 6091. Many of the I/O devices 6092, communication devices 6086, and the data storage unit 6088 may reside on the motherboard 6005 while the keyboard 6082 and the mouse 6084 may be add-on peripherals. In other examples, some or all the I/O devices 6092, communication devices 6086, and the data storage unit 6088 are add-on peripherals and do not reside on the motherboard 6005.

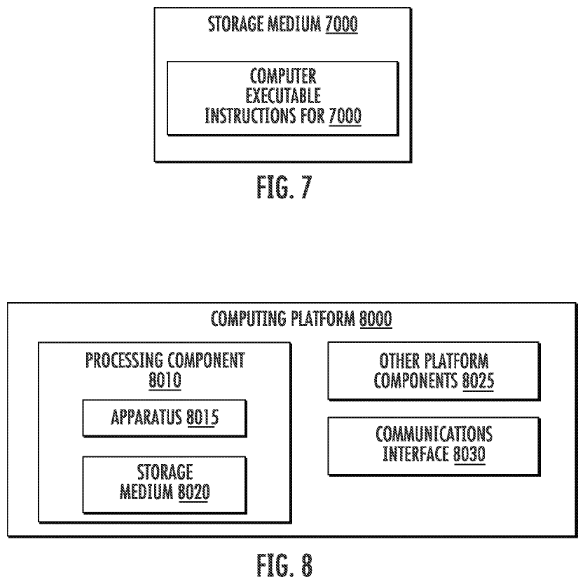

FIG. 7 illustrates an example of a storage medium 7000 to store processor data structures. Storage medium 7000 may include an article of manufacture. In some examples, storage medium 7000 may include any non-transitory computer readable medium or machine-readable medium, such as an optical, magnetic or semiconductor storage. Storage medium 7000 may store various types of computer executable instructions, such as instructions to implement logic flows and/or techniques described herein. Examples of a computer readable or machine-readable storage medium may include any tangible media capable of storing electronic data, including volatile memory or non-volatile memory, removable or non-removable memory, erasable or non-erasable memory, writeable or re-writeable memory, and so forth. Examples of computer executable instructions may include any suitable type of code, such as source code, compiled code, interpreted code, executable code, static code, dynamic code, object-oriented code, visual code, and the like. The examples are not limited in this context.

FIG. 8 illustrates an example computing platform 8000. In some examples, as shown in FIG. 8, computing platform 8000 may include a processing component 8010, other platform components or a communications interface 8030. According to some examples, computing platform 8000 may be implemented in a computing device such as a server in a system such as a data center or server farm that supports a manager or controller for managing configurable computing resources. Furthermore, the communications interface 8030 may include a wake-up radio (WUR) and may can wake up a main radio of the computing platform 8000.

According to some examples, processing component 8010 may execute processing operations or logic for apparatus 8015 described herein such as the correction logic circuitry discussed in conjunction with FIGS. 1-7. Processing component 8010 may include various hardware elements, software elements, or a combination of both. Examples of hardware elements may include devices, logic devices, components, processors, microprocessors, circuits, processor circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), memory units, logic gates, registers, semiconductor device, chips, microchips, chip sets, and so forth. Examples of software elements, which may reside in the storage medium 8020, may include software components, programs, applications, computer programs, application programs, device drivers, system programs, software development programs, machine programs, operating system software, middleware, firmware, software modules, routines, subroutines, functions, methods, procedures, software interfaces, application program interfaces (API), instruction sets, computing code, computer code, code segments, computer code segments, words, values, symbols, or any combination thereof. Determining whether an example is implemented using hardware elements and/or software elements may vary in accordance with any number of factors, such as desired computational rate, power levels, heat tolerances, processing cycle budget, input data rates, output data rates, memory resources, data bus speeds and other design or performance constraints, as desired for a given example.

In some examples, other platform components 8025 may include common computing elements, such as one or more processors, multi-core processors, co-processors, memory units, chipsets, controllers, peripherals, interfaces, oscillators, timing devices, video cards, audio cards, multimedia input/output (I/O) components (e.g., digital displays), power supplies, and so forth. Examples of memory units may include without limitation various types of computer readable and machine readable storage media in the form of one or more higher speed memory units, such as read-only memory (ROM), random-access memory (RAM), dynamic RAM (DRAM), Double-Data-Rate DRAM (DDRAM), synchronous DRAM (SDRAM), static RAM (SRAM), programmable ROM (PROM), erasable programmable ROM (EPROM), electrically erasable programmable ROM (EEPROM), flash memory, polymer memory such as ferroelectric polymer memory, ovonic memory, phase change or ferroelectric memory, silicon-oxide-nitride-oxide-silicon (SONOS) memory, magnetic or optical cards, an array of devices such as Redundant Array of Independent Disks (RAID) drives, solid state memory devices (e.g., USB memory), solid state drives (SSD) and any other type of storage media suitable for storing information.

In some examples, communications interface 8030 may include logic and/or features to support a communication interface. For these examples, communications interface 8030 may include one or more communication interfaces that operate according to various communication protocols or standards to communicate over direct or network communication links. Direct communications may occur via use of communication protocols or standards described in one or more industry standards (including progenies and variants) such as those associated with the PCI Express specification. Network communications may occur via use of communication protocols or standards such as those described in one or more Ethernet standards promulgated by the Institute of Electrical and Electronics Engineers (IEEE). For example, one such Ethernet standard may include IEEE 802.3-2012, Carrier sense Multiple access with Collision Detection (CSMA/CD) Access Method and Physical Layer Specifications, Published in December 2012 (hereinafter “IEEE 802.3”). Network communication may also occur according to one or more OpenFlow specifications such as the OpenFlow Hardware Abstraction API Specification. Network communications may also occur according to Infiniband Architecture Specification, Volume 1, Release 1.3, published in March 2015 (“the Infiniband Architecture specification”).

Computing platform 8000 may be part of a computing device that may be, for example, a server, a server array or server farm, a web server, a network server, an Internet server, a workstation, a mini-computer, a main frame computer, a supercomputer, a network appliance, a web appliance, a distributed computing system, multiprocessor systems, processor-based systems, or combination thereof. Accordingly, functions and/or specific configurations of computing platform 8000 described herein, may be included or omitted in various examples of computing platform 8000, as suitably desired.

The components and features of computing platform 8000 may be implemented using any combination of discrete circuitry, ASICs, logic gates and/or single chip architectures. Further, the features of computing platform 8000 may be implemented using microcontrollers, programmable logic arrays and/or microprocessors or any combination of the foregoing where suitably appropriate. It is noted that hardware, firmware and/or software elements may be collectively or individually referred to herein as “logic”.

It should be appreciated that the exemplary computing platform 8000 shown in the block diagram of FIG. 8 may represent one functionally descriptive example of many potential implementations. Accordingly, division, omission or inclusion of block functions depicted in the accompanying figures does not infer that the hardware components, circuits, software and/or elements for implementing these functions would necessarily be divided, omitted, or included in examples.

One or more features of at least one example may be implemented by representative instructions stored on at least one machine-readable medium which represents various logic within the processor, which when read by a machine, computing device or system causes the machine, computing device or system to fabricate logic to perform the techniques described herein. Such representations, known as “IP cores”, may be stored on a tangible, machine readable medium and supplied to various customers or manufacturing facilities to load into the fabrication machines that actually make the logic or processor.

Various examples may be implemented using hardware elements, software elements, or a combination of both. In some examples, hardware elements may include devices, components, processors, microprocessors, circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), memory units, logic gates, registers, semiconductor device, chips, microchips, chip sets, and so forth. In some examples, software elements may include software components, programs, applications, computer programs, application programs, system programs, machine programs, operating system software, middleware, firmware, software modules, routines, subroutines, functions, methods, procedures, software interfaces, application program interfaces (API), instruction sets, computing code, computer code, code segments, computer code segments, words, values, symbols, or any combination thereof. Determining whether an example is implemented using hardware elements and/or software elements may vary in accordance with any number of factors, such as desired computational rate, power levels, heat tolerances, processing cycle budget, input data rates, output data rates, memory resources, data bus speeds and other design or performance constraints, as desired for a given implementation.

Some examples may include an article of manufacture or at least one computer-readable medium. A computer-readable medium may include a non-transitory storage medium to store logic. In some examples, the non-transitory storage medium may include one or more types of computer-readable storage media capable of storing electronic data, including volatile memory or non-volatile memory, removable or non-removable memory, erasable or non-erasable memory, writeable or re-writeable memory, and so forth. In some examples, the logic may include various software elements, such as software components, programs, applications, computer programs, application programs, system programs, machine programs, operating system software, middleware, firmware, software modules, routines, subroutines, functions, methods, procedures, software interfaces, API, instruction sets, computing code, computer code, code segments, computer code segments, words, values, symbols, or any combination thereof.

According to some examples, a computer-readable medium may include a non-transitory storage medium to store or maintain instructions that when executed by a machine, computing device or system, cause the machine, computing device or system to perform methods and/or operations in accordance with the described examples. The instructions may include any suitable type of code, such as source code, compiled code, interpreted code, executable code, static code, dynamic code, and the like. The instructions may be implemented according to a predefined computer language, manner or syntax, for instructing a machine, computing device or system to perform a certain function. The instructions may be implemented using any suitable high-level, low-level, object-oriented, visual, compiled and/or interpreted programming language.

Some examples may be described using the expression “in one example” or “an example” along with their derivatives. These terms mean that a particular feature, structure, or characteristic described in connection with the example is included in at least one example. The appearances of the phrase “in one example” in various places in the specification are not necessarily all referring to the same example.

Some examples may be described using the expression “coupled” and “connected” along with their derivatives. These terms are not necessarily intended as synonyms for each other. For example, descriptions using the terms “connected” and/or “coupled” may indicate that two or more elements are in direct physical or electrical contact with each other. The term “coupled,” however, may also mean that two or more elements are not in direct contact with each other, but yet still co-operate or interact with each other.

In addition, in the foregoing Detailed Description, it can be seen that various features are grouped together in a single example for the purpose of streamlining the disclosure. This method of disclosure is not to be interpreted as reflecting an intention that the claimed examples require more features than are expressly recited in each claim. Rather, as the following claims reflect, inventive subject matter lies in less than all features of a single disclosed example. Thus, the following claims are hereby incorporated into the Detailed Description, with each claim standing on its own as a separate example. In the appended claims, the terms “including” and “in which” are used as the plain-English equivalents of the respective terms “including” and “wherein,” respectively. Moreover, the terms “first,” “second,” “third,” and so forth, are used merely as labels, and are not intended to impose numerical requirements on their objects.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

A data processing system suitable for storing and/or executing program code will include at least one processor coupled directly or indirectly to memory elements through a system bus. The memory elements can include local memory employed during actual execution of the program code, bulk storage, and cache memories which provide temporary storage of at least some program code to reduce the number of times code must be retrieved from bulk storage during execution. The term “code” covers a broad range of software components and constructs, including applications, drivers, processes, routines, methods, modules, firmware, microcode, and subprograms. Thus, the term “code” may be used to refer to any collection of instructions which, when executed by a processing system, perform a desired operation or operations.

Logic circuitry, devices, and interfaces herein described may perform functions implemented in hardware and also implemented with code executed on one or more processors. Logic circuitry refers to the hardware or the hardware and code that implements one or more logical functions. Circuitry is hardware and may refer to one or more circuits. Each circuit may perform a particular function. A circuit of the circuitry may include discrete electrical components interconnected with one or more conductors, an integrated circuit, a chip package, a chip set, memory, or the like. Integrated circuits include circuits created on a substrate such as a silicon wafer and may include components. And integrated circuits, processor packages, chip packages, and chipsets may include one or more processors.

Processors may receive signals such as instructions and/or data at the input(s) and process the signals to generate the at least one output. While executing code, the code changes the physical states and characteristics of transistors that make up a processor pipeline. The physical states of the transistors translate into logical bits of ones and zeros stored in registers within the processor. The processor can transfer the physical states of the transistors into registers and transfer the physical states of the transistors to another storage medium.

A processor may include circuits to perform one or more sub-functions implemented to perform the overall function of the processor. One example of a processor is a state machine or an application-specific integrated circuit (ASIC) that includes at least one input and at least one output. A state machine may manipulate the at least one input to generate the at least one output by performing a predetermined series of serial and/or parallel manipulations or transformations on the at least one input.

While the present examples have described one or more features for use in an in-line motorized strut, it is envisioned that the one or more features may be used in a motorized strut having an offset motor design (e.g., longitudinal axis of the motor is offset from the longitudinal axis of the threaded rod). For example, by incorporating a secondary telescoping mechanism into a motorized strut having an offset motor design, the motorized strut could benefit from having a larger working length, meaning less strut changeouts and less inventory. As such, the present disclosure should not be limited to an in-line design unless specifically claimed

Thus arranged, in accordance with the features of the present disclosure, the motorized struts serve to maximize the range (e.g., working range) of a motorized strut, and more preferably an in-line motorized strut. In use, the addition of an independent telescoping member allows quick, manual length adjustment in, for example, the operating room during initial setup, while not using any of the working length associated with rotation of the threaded rod. In addition, and/or alternatively, incorporation of a two-stage telescoping design allows for essentially twice the working length of a motorized strut. If combined, a motorized strut having a larger working length (e.g., 2× working range) and the capability to be manually lengthened in the operating room without using any of the working length can be provided

While the present disclosure refers to certain examples, numerous modifications,

alterations, and changes to the described examples are possible without departing from the sphere and scope of the present disclosure, as defined in the appended claim(s). Accordingly, it is intended that the present disclosure not be limited to the described examples, but that it has the full scope defined by the language of the following claims, and equivalents thereof. The discussion of any example is meant only to be explanatory and is not intended to suggest that the scope of the disclosure, including the claims, is limited to these examples. In other words, while illustrative examples of the disclosure have been described in detail herein, it is to be understood that the inventive concepts may be otherwise variously embodied and employed, and that the appended claims are intended to be construed to include such variations, except as limited by the prior art.

The foregoing discussion has been presented for purposes of illustration and description and is not intended to limit the disclosure to the form or forms disclosed herein. For example, various features of the disclosure are grouped together in one or more examples or configurations for the purpose of streamlining the disclosure. However, it should be understood that various features of the certain examples or configurations of the disclosure may be combined in alternate examples, or configurations. Any example or feature of any section, portion, or any other component shown or particularly described in relation to various examples of similar sections, portions, or components herein may be interchangeably applied to any other similar example or feature shown or described herein. Additionally, components with the same name may be the same or different, and one of ordinary skill in the art would understand each component could be modified in a similar fashion or substituted to perform the same function.

As used herein, an element or step recited in the singular and proceeded with the word “a” or “an” should be understood as not excluding plural elements or steps, unless such exclusion is explicitly recited. Furthermore, references to “one example” of the present disclosure are not intended to be interpreted as excluding the existence of additional examples that also incorporate the recited features.

The phrases “at least one”, “one or more”, and “and/or”, as used herein, are open-ended expressions that are both conjunctive and disjunctive in operation. The terms “a” (or “an”), “one or more” and “at least one” can be used interchangeably herein. All directional references (e.g., proximal, distal, upper, lower, upward, downward, left, right, lateral, longitudinal, front, back, top, bottom, above, below, vertical, horizontal, radial, axial, clockwise, and counterclockwise) are only used for identification purposes to aid the reader's understanding of the present disclosure, and do not create limitations, particularly as to the position, orientation, or use of this disclosure. Connection references (e.g., engaged, attached, coupled, connected, and joined) are to be construed broadly and may include intermediate members between a collection of elements and relative to movement between elements unless otherwise indicated. As such, connection references do not necessarily infer that two elements are directly connected and in fixed relation to each other. All rotational references describe relative movement between the various elements. Identification references (e.g., primary, secondary, first, second, third, fourth, etc.) are not intended to connote importance or priority but are used to distinguish one feature from another. The drawings are for purposes of illustration only and the dimensions, positions, order and relative to sizes reflected in the drawings attached hereto may vary.

### FURTHER EXAMPLES

The following are further examples. Specifics in the examples may be used anywhere in one or more of the examples above.

Example 1 is a method for a graphical user interface to adjust a correction path of a prescription for a bone fixator, the method comprising providing the prescription which defines scheduled adjustments of the bone fixator and determines the correction path and a correction rate of a moving bone segment connected to the bone fixator relative to a fixed bone segment also connected to the bone fixator; generating a display of an image of the fixed bone segment and the moving bone segment according to the prescription; interacting with a user via a first user interface element to select a point on the correction path of the prescription; adjusting a path of the moving bone segment from the correction path of the prescription via user actions and generating a modified prescription accounting for a modified correction path, the user actions comprising at least one of dragging a selected point on the correction path of the prescription to identify a new waypoint to adjust a translation, an angulation, or a combination of the translation and the angulation of the moving bone segment; or entering keystrokes to adjust the translation, the angulation, or a combination of the translation and the angulation of the moving bone segment to the new waypoint. In Example 2, the method of Example 1, further comprising a second user interface element to interact with the user to select a perspective view of the display, wherein the image of the fixed bone segment and the moving bone segment comprises two dimensional radiological images of the bone segments connected with the bone fixator, wherein the image of the fixed bone segment and the moving bone segment comprises a three-dimensional image of the bone segments connected with the bone fixator, or wherein the image of the fixed bone segment and the moving bone segment comprises a bone model defined by user input. In Example 3, the method of Example 1, further comprising a third user interface element to describe a remaining deformity in response to identification of the new waypoint. In Example 4, the method of Example 1, further comprising a fourth user interface element to describe hardware adjustments responsive to identification of the new waypoint, wherein the correction path is limited to hardware constraints In Example 5, the method of Example 1, further comprising a fifth user interface element to describe a number of days associated with the prescription, wherein the number of days is updated responsive to identification of the new waypoint in accordance with the modified prescription accounting, the new waypoint to update a final corrected state, wherein a partial deformity remains after the update to the final corrected state, wherein a final prescription includes the partial deformity, wherein the final prescription includes additional days of adjustment to correct the partial deformity. In Example 6, the method of Example 1, wherein adjustment of a final point on the correction path results in an over-correction or an under-correction of one of more components of the translational deformity recorded in the modified prescription accounting. In Example 7, the method of Example 1, wherein adjustment of the angulation of the moving bone segment beyond the angulation of the prescription results in an over-correction or an under-correction of one of more components of the angular deformity recorded in the modified prescription accounting. In Example 8, the method of Example 1, wherein translational and angular corrections of the moving bone segment are grouped into one or more correction stages, wherein all deformity component corrections within a correction stage, of the one or more correction stages, occur simultaneously according to associated maximum correction rates. In Example 9, the method of any one or more of Examples 1-8, wherein the image comprises a three-dimensional model of the bone segments, a statistical model, or a three-dimensional medical image, wherein the three-dimensional medical model is based on a magnetic resonance image (MRI), a computerized tomography (CT) scan, AP and LAT radiographs, other x-ray images, other medical images, or a combination thereof; further comprising calculating three-dimensional projection of two curves on two-dimensional images to generate one three-dimensional linear curve for adjustments of the correction path or generating adjustments based on user input of a nonlinear three-dimensional curve to generate three-dimensional linear adjustments for the correction path.

Example 10 is a computer-readable storage medium, comprising a plurality of instructions for a code block interface to adjust a correction path of a prescription for a bone fixator to correct a deformity of bone segments, that when executed by processing circuitry, enable processing circuitry to display a set of code blocks for the prescription, wherein each code block describes a magnitude, a direction, and a maximum correction rate of translational and angular deformity components of the prescription; interact with a user to modify the magnitude, the direction, the maximum correction rate, or any combination of the magnitude, the direction, and the maximum correction rate of the code blocks for the prescription, wherein modifying the magnitude or the direction of one or more of the code blocks results in generation of one or more new code blocks in the set of the code blocks to account for remaining deformity components of the prescription; interact with the user to sort the set of the code blocks into one or more correction stages, wherein the code blocks associated with a correction stage are corrected simultaneously by the prescription in accordance with a respective maximum correction rate associated with each of the code blocks; and generate a modified prescription accounting for changes to the magnitudes, the directions, the maximum correction rates, and the correction orders of the code blocks. In Example 11, the computer-readable storage medium of Example 10, wherein any remaining code blocks not assigned to the one or more correction stages are excluded. In Example 12, the computer-readable storage medium of Example 10, wherein the processing circuitry is further enabled to generate a display of an image of a fixed bone segment and a moving bone segment. In Example 13, the computer-readable storage medium of Example 12, wherein generation of the display comprises generation of the display with two images, a first image of a state of the deformity prior to correction by a selected stage and second image of a state of the deformity after correction by the selected stage. In Example 14, the computer-readable storage medium of Example 12, wherein the image comprises a three-dimensional model of the bone segments, a statistical model, or a three-dimensional medical image, wherein the three-dimensional medical model is based on a magnetic resonance image (MRI), a computerized tomography (CT) scan, AP and LAT radiographs, other x-ray images, other medical images, or a combination thereof, further comprising calculating one three-dimensional linear curve for adjustments of the correction path. In Example 15, the computer-readable storage medium of Example 10, wherein the code blocks are assigned individual maximum correction rates or assume a maximum correction rate associated with at least one of the one or more correction stages. In Example 16, the computer-readable storage medium of any one or more of Examples 10-15, the interacting with the user to update a final corrected state, wherein a partial deformity remains after the update to the final corrected state, wherein a final prescription includes the partial deformity, wherein the final prescription includes additional days of adjustment to correct the partial deformity, wherein the correction path is limited to hardware constraints.

Example 17 is an apparatus to adjust a correction path of a prescription for a bone fixator, the apparatus comprising a memory; and logic circuitry coupled with the memory to perform operations to provide the prescription which defines scheduled adjustments of the bone fixator and determines the correction path and a correction rate of a moving bone segment connected to the bone fixator relative to a fixed bone segment also connected to the bone fixator; generating a display of an image of the fixed bone segment and the moving bone segment according the prescription; interacting with a user via a first user interface element to select a point on the correction path of the prescription; adjusting a path of the moving bone segment from the correction path of the prescription via user actions and generating a modified prescription accounting for a modified correction path, the user actions comprising at least one of dragging a selected point on the correction path of the prescription to identify a new waypoint to adjust a translation, an angulation, or a combination of the translation and the angulation of the moving bone segment; or entering keystrokes to adjust the translation, the angulation, or a combination of the translation and the angulation of the moving bone segment to the new waypoint. In Example 18, the apparatus of Example 17, the operations further to display a set of code blocks for the prescription, wherein each code block describes a magnitude, a direction, and a maximum correction rate of translational and angular deformity components of the prescription; interact with the user to modify the magnitude, the direction, the maximum correction rate, or any combination of the magnitude, the direction, and the maximum correction rate of the code blocks for the prescription, wherein modifying the magnitude or the direction of one or more of the code blocks results in generation of one or more new code blocks to account for remaining deformity components of the prescription; interact with the user to sort the code blocks into one or more correction stages, wherein the code blocks within a correction stage are corrected simultaneously by the prescription according to a respective maximum correction rate for each of the code blocks within the correction stage; generate a modified prescription accounting for changes to the magnitudes, the directions, the maximum correction rates, and the correction orders of the code blocks. In Example 19, the apparatus of Example 18, the operations further to calculate three-dimensional projection of two curves on two-dimensional images to generate one three-dimensional linear curve for adjustments of the correction path, generate adjustments based on user input of a nonlinear three-dimensional curve to generate three-dimensional linear adjustments for the correction path, or a combination thereof, wherein the correction path is limited to hardware constraints. In Example 20, the apparatus of Example 19, the operations further to interact via a second user interface element with the user to select a perspective view of the display, interact via a third user interface element to describe the remaining deformity in response to identification of the new waypoint, and interact via a fourth user interface element to describe a number of days associated with the prescription, wherein the number of days is updated responsive to identification of the new waypoint. In Example 21, the apparatus of Example 20, the operations further to interact via a fifth user interface element with the user to describe hardware adjustments responsive to identification of the new waypoint. In Example 22, the apparatus of Example 19, wherein a final prescription includes a partial deformity, the interacting with the user to update a final corrected state, wherein the partial deformity remains after the update to the final corrected state, wherein the final prescription includes additional days of adjustment to correct the partial deformity.

1. A method for a graphical user interface to adjust a correction path of a prescription for a bone fixator, the method comprising:
providing the prescription which defines scheduled adjustments of the bone fixator and determines the correction path and a correction rate of a moving bone segment connected to the bone fixator relative to a fixed bone segment also connected to the bone fixator;
generating a display of an image of the fixed bone segment and the moving bone segment according to the prescription;
interacting with a user via a first user interface element to select a point on the correction path of the prescription; and
adjusting a path of the moving bone segment from the correction path of the prescription via user actions and generating a modified prescription accounting for a modified correction path, the user actions comprising at least one of dragging a selected point on the correction path of the prescription to identify a new waypoint to adjust a translation, an angulation, or a combination of the translation and the angulation of the moving bone segment; or entering keystrokes to adjust the translation, the angulation, or a combination of the translation and the angulation of the moving bone segment to the new waypoint.

2. The method of claim 1, further comprising a second user interface element to interact with the user to select a perspective view of the display, wherein the image of the fixed bone segment and the moving bone segment comprises two dimensional radiological images of the bone segments connected with the bone fixator, wherein the image of the fixed bone segment and the moving bone segment comprises a three-dimensional image of the bone segments connected with the bone fixator, or wherein the image of the fixed bone segment and the moving bone segment comprises a bone model defined by user input.

3. The method of claim 1, further comprising a third user interface element to describe a remaining deformity in response to identification of the new waypoint.

4. The method of claim 1, further comprising a fourth user interface element to describe hardware adjustments responsive to identification of the new waypoint, wherein the correction path is limited to hardware constraints.

5. The method of claim 1, further comprising a fifth user interface element to describe a number of days associated with the prescription, wherein the number of days is updated responsive to identification of the new waypoint in accordance with the modified prescription accounting, the new waypoint to update a final corrected state, wherein a partial deformity remains after the update to the final corrected state, wherein a final prescription includes the partial deformity, wherein the final prescription includes additional days of adjustment to correct the partial deformity.

6. The method of claim 1, wherein adjustment of a final point on the correction path results in an over-correction or an under-correction of one of more components of the translational deformity recorded in the modified prescription accounting.

7. The method of claim 1, wherein adjustment of the angulation of the moving bone segment beyond the angulation of the prescription results in an over-correction or an under-correction of one of more components of the angular deformity recorded in the modified prescription accounting.

8. The method of claim 1, wherein translational and angular corrections of the moving bone segment are grouped into one or more correction stages, wherein all deformity component corrections within a correction stage, of the one or more correction stages, occur simultaneously according to associated maximum correction rates.

9. The method of claim 1, wherein the image comprises a three-dimensional model of the bone segments, a statistical model, or a three-dimensional medical image, wherein the three-dimensional medical model is based on a magnetic resonance image (MRI), a computerized tomography (CT) scan, AP and LAT radiographs, other x-ray images, other medical images, or a combination thereof; further comprising calculating three-dimensional projection of two curves on two-dimensional images to generate one three-dimensional linear curve for adjustments of the correction path or generating adjustments based on user input of a nonlinear three-dimensional curve to generate three-dimensional linear adjustments for the correction path.

10. A computer-readable storage medium, comprising a plurality of instructions for a code block interface to adjust a correction path of a prescription for a bone fixator to correct a deformity of bone segments, that when executed by processing circuitry, enable processing circuitry to:
display a set of code blocks for the prescription, wherein each code block describes a magnitude, a direction, and a maximum correction rate of translational and angular deformity components of the prescription;
interact with a user to modify the magnitude, the direction, the maximum correction rate, or any combination of the magnitude, the direction, and the maximum correction rate of the code blocks for the prescription, wherein modifying the magnitude or the direction of one or more of the code blocks results in generation of one or more new code blocks in the set of the code blocks to account for remaining deformity components of the prescription;
interact with the user to sort the set of the code blocks into one or more correction stages, wherein the code blocks associated with a correction stage are corrected simultaneously by the prescription in accordance with a respective maximum correction rate associated with each of the code blocks; and
generate a modified prescription accounting for changes to the magnitudes, the directions, the maximum correction rates, and the correction orders of the code blocks.

11. The computer-readable storage medium of claim 10, wherein any remaining code blocks not assigned to the one or more correction stages are excluded.

12. The computer-readable storage medium of claim 10, wherein the processing circuitry is further enabled to generate a display of an image of a fixed bone segment and a moving bone segment.

13. The computer-readable storage medium of claim 12, wherein generation of the display comprises generation of the display with two images, a first image of a state of the deformity prior to correction by a selected stage and second image of a state of the deformity after correction by the selected stage.

14. The computer-readable storage medium of claim 12, wherein the image comprises a three-dimensional model of the bone segments, a statistical model, or a three-dimensional medical image, wherein the three-dimensional medical model is based on a magnetic resonance image (MRI), a computerized tomography (CT) scan, AP and LAT radiographs, other x-ray images, other medical images, or a combination thereof, further comprising calculating one three-dimensional linear curve for adjustments of the correction path.

15. The computer-readable storage medium of claim 10, wherein the code blocks are assigned individual maximum correction rates or assume a maximum correction rate associated with at least one of the one or more correction stages.

16. The computer-readable storage medium of claim 10, the interacting with the user to update a final corrected state, wherein a partial deformity remains after the update to the final corrected state, wherein a final prescription includes the partial deformity, wherein the final prescription includes additional days of adjustment to correct the partial deformity, wherein the correction path is limited to hardware constraints.

17. An apparatus to adjust a correction path of a prescription for a bone fixator, the apparatus comprising:
a memory; and
logic circuitry coupled with the memory to perform operations to:
provide the prescription which defines scheduled adjustments of the bone fixator and determines the correction path and a correction rate of a moving bone segment connected to the bone fixator relative to a fixed bone segment also connected to the bone fixator;
generating a display of an image of the fixed bone segment and the moving bone segment according to the prescription;
interacting with a user via a first user interface element to select a point on the correction path of the prescription; and
adjusting a path of the moving bone segment from the correction path of the prescription via user actions and generating a modified prescription accounting for a modified correction path, the user actions comprising at least one of dragging a selected point on the correction path of the prescription to identify a new waypoint to adjust a translation, an angulation, or a combination of the translation and the angulation of the moving bone segment; or entering keystrokes to adjust the translation, the angulation, or a combination of the translation and the angulation of the moving bone segment to the new waypoint.

18. The apparatus of claim 17, the operations further to:
display a set of code blocks for the prescription, wherein each code block describes a magnitude, a direction, and a maximum correction rate of translational and angular deformity components of the prescription;
interact with the user to modify the magnitude, the direction, the maximum correction rate, or any combination of the magnitude, the direction, and the maximum correction rate of the code blocks for the prescription, wherein modifying the magnitude or the direction of one or more of the code blocks results in generation of one or more new code blocks to account for remaining deformity components of the prescription;
interact with the user to sort the code blocks into one or more correction stages, wherein the code blocks within a correction stage are corrected simultaneously by the prescription according to a respective maximum correction rate for each of the code blocks within the correction stage; and
generate a modified prescription accounting for changes to the magnitudes, the directions, the maximum correction rates, and the correction orders of the code blocks.

19. The apparatus of claim 18, the operations further to calculate three-dimensional projection of two curves on two-dimensional images to generate one three-dimensional linear curve for adjustments of the correction path, generate adjustments based on user input of a nonlinear three-dimensional curve to generate three-dimensional linear adjustments for the correction path, or a combination thereof, wherein the correction path is limited to hardware constraints.

20. The apparatus of claim 19, the operations further to interact via a second user interface element with the user to select a perspective view of the display, interact via a third user interface element to describe the remaining deformity in response to identification of the new waypoint, and interact via a fourth user interface element to describe a number of days associated with the prescription, wherein the number of days is updated responsive to identification of the new waypoint.

21. The apparatus of claim 20, the operations further to interact via a fifth user interface element with the user to describe hardware adjustments responsive to identification of the new waypoint.

22. The apparatus of claim 19, wherein a final prescription includes a partial deformity, the interacting with the user to update a final corrected state, wherein the partial deformity remains after the update to the final corrected state, wherein the final prescription includes additional days of adjustment to correct the partial deformity.
